(19) United States
(12) Patent Application Publication
(10) Pub. No.: US 2006/0148818 A1
(43) Pub. Date: Jul. 6, 2006

(54) Novel tetraydrospiro(piperdine-2,7'- pyrrolo{3,2-b}pyridine derivatives and novel in-dole derivatives useful in the treatment of 5-ht6 receptor-related disorders

(76) Inventors: Gary Johansson, Uppsala (SE); Peter Brandt, Solna (SE); Graeme Dykes, Uppsala (SE); Bjorn M Nilsson, Stockholm (SE)

Correspondence Address: FISH & RICHARDSON PC, P.O. BOX 1022, MINNEAPOLIS, MN, 55440-1022, US

(21) Appl. No.: 10/536,603

(22) PCT Filed: Oct. 20, 2004
(86) PCT No.: PCT/SE04/01508

§ 371 Date: Dec. 20, 2005

(30) Foreign Application Priority Data
Oct. 20, 2003 (SE) 03027604
Nov. 18, 2003 (US) 60523126

(51) Int. Cl. A61K 31/496; A61K 31/4747; A61K 31/4439; A61K 31/405; C07D 471/10; C07D 403/02

(52) U.S. Cl. 514/254.09; 514/278; 514/339; 514/415; 514/411; 544/373; 546/16; 546/277.4; 548/495

(57) ABSTRACT

The present invention relates to compounds of formula (I): Formula (I) wherein U, P, W₁, W₂, W₃, v, Y, Z, R^{m}, and R^{m′} are as described herein, to pharmaceutical compositions comprising the compounds, to processes for their preparation, as well as to the use of the compounds for the preparation of a medicament against 5-HT₆ receptor-related disorders.

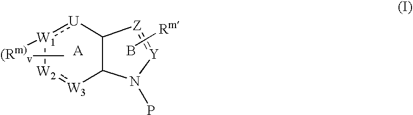

### TECHNICAL FIELD

The present invention relates to novel compounds, to pharmaceutical compositions comprising the compounds, to processes for their preparation, as well as to the use of the compounds for the preparation of a medicament against 5-HT₆ receptor-related disorders.

### BACKGROUND OF THE INVENTION

Obesity is a condition characterized by an increase in body fat content resulting in excess body weight above accepted norms. Obesity is the most important nutritional disorder in the western world and represents a major health problem in all industrialized countries. This disorder leads to increased mortality due to increased incidences of diseases such as cardiovascular disease, digestive disease, respiratory disease, cancer and type 2 diabetes. Searching for compounds, which reduce body weight has been going on for many decades. One line of research has been activation of serotoninergic systems, either by direct activation of serotonin receptor subtypes or by inhibiting serotonin reuptake. The exact receptor subtype profile required is however not known.

Serotonin (5-hydroxytryptamine or 5-HT), a key transmitter of the peripheral and central nervous system, modulates a wide range of physiological and pathological functions, including anxiety, sleep regulation, aggression, feeding and depression. Multiple serotonin receptor subtypes have been identified and cloned. One of these, the 5-HT₆ receptor, was cloned by several groups in 1993 (Ruat, M. et al. (1993) Biochem. Biophys. Res. Commun. 193: 268-276; Sebben, M. et al. (1994) NeuroReport 5: 2553-2557). This receptor is positively coupled to adenylyl cyclase and displays affinity for antidepressants such as clozapine. Recently, the effect of 5-HT₆ antagonist and 5-HT₆ antisense oligonucleotides to reduce food intake in rats has been reported (Bentley, J. C. et al. (1999) Br J Pharmacol. Suppl. 126, P66; Bentley, J. C. et al. (1997) J. Psychopharmacol. Suppl. A64, 255; Woolley M. L. et al. (2001) Neuropharmacology 41: 210-219).

Compounds with enhanced affinity and selectivity for the 5-HT₆ receptor have been identified, e.g. in WO 00/34242 and by Isaac, M. et al. (2000) 6-Bicyclopiperazinyl-1-arylsulphonylindoles and 6-Bicyclopiperidinyl-1-arylsulphonylindoles derivatives as novel, potent and selective 5-HT₆ receptor antagonists. Bioorganic & Medicinal Chemistry Letters 10: 1719-1721 (2000), Bioorganic & Medicinal Chemistry Letters 13: 3355-3359 (2003), Expert Opinion Therapeutic Patents 12(4) 513-527 (2002).

It has surprisingly been found that the compounds according to the present invention show affinity for the 5-HT₆ receptor as antagonists at nanomolar range. Compounds according to the present invention and their pharmaceutically acceptable salts have 5-HT₆ receptor antagonist, agonist and partial agonist activity and are believed to be of potential use in the treatment or prophylaxis of obesity and type 2 diabetes, to achieve reduction of body weight and of body weight gain, as well as in the treatment or prophylaxis of disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea and/or schizophrenia, panic attacks, Attention Deficit Hyperactive Disorder (ADHD), withdrawal from drug abuse, neurodegenerative diseases characterized by impaired neuronal growth, and pain. The reduction of body weight and of body weight gain (e.g. treating body-weight disorders) is achieved inter alia by reduction of food intake. As used herein, the term “body weight disorders” refers to the disorders caused by an imbalance between energy intake and energy expenditure, resulting in abnormal (e.g., excessive) body weight. Such body weight-disorders include obesity.

### INFORMATION DISCLOSURE

WO 99/42465 discloses sulphonamides derivatives that bind to the 5-HT₆ receptor and that can be used for the treatment of CNS disorders such as anxiety, depression, epilepsy, obsessive compulsive disorders, cognitive disorders, ADHD, anorexia and bulimia, schizophrenia, and drug abuse.

WO 01/32646 A1 discloses compounds that bind to the 5-HT₆ receptor and that are used for the treatment of CNS disorders and which inter alia may be used for the treatment of eating disorders.

WO 99/37623 A2 discloses compounds that bind to the 5-HT₆ receptor and that are used for the treatment of CNS disorders and which inter alia may be used for the treatment of eating disorders.

WO 99/42465 A3 discloses compounds that bind to the 5-HT₆ receptor and that are used for the treatment of CNS disorders and which inter alia may be used for the treatment of eating disorders.

EP 0 815 861 A1 discloses compounds that bind to the 5-HT₆ receptor and that are used for the treatment of CNS disorders.

WO 99/02502 A2 discloses compounds that bind to the 5-HT₆ receptor and that are used for the treatment of CNS disorders and which inter alia may be used for the treatment of eating disorders.

WO 98/27081 A1 discloses compounds that bind to the 5-HT₆ receptor and that are used for the treatment of CNS disorders and which inter alia may be used for the treatment of eating disorders.

EP 0701819 discloses compounds that bind to the 5-HT_{1D} receptor and that are used for the treatment of CNS disorders and obesity.

U.S. Pat. No. 6,191,141 and WO 01/12629 disclose compounds that bind to the 5-HT₆ receptor and that are used for the treatment of CNS disorders.

WO03/072198 disclose benzenesulphonamide derivatives for the treatment of obesity.

No publications disclose the compounds and their use according to the present invention against 5-HT₆ receptor-related disorders.

### DISCLOSURE OF THE INVENTION

One object of the present invention is a compound of the Formula (I) 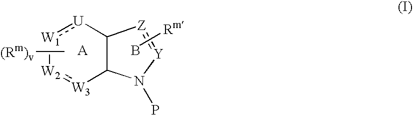
wherein:
v is 1 or 2 and P is selected from a substituent of Formula (II) and Formula (II); 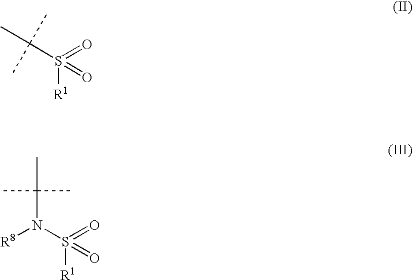
or P may also be selected from H or C₁₋₆-alkyl provided that R^{m} is selected from —NHSO₂R¹¹, —SO₂NR⁸R¹¹ or —S(O)ₑR¹¹, wherein R¹¹ is selected from aryl and heteroaryl and where e is 0, 1, 2 or 3, v is 1 and R^{m′} is H; [symbol]
represents a single bond or a double bond, with the proviso that both [symbol]
represent double bonds or that both [symbol] represent single bonds;
W₁, W₂, W₃, Z and Y are each a carbon atom; or
one of W₁, W₂, W₃, Z and Y is a nitrogen atom, while the remainder being carbon atoms, provided that both [symbol] in Formula (I) represent single bonds;
U is selected from CHR⁴, CR⁴ and CR⁴R^{4′}, provided that when the dotted line connecting W₁ and U is a double bond, then U is CR⁴; and further provided that when the dotted line connecting W₁ and U is a single bond, then U is selected from CHR⁴ and CR⁴R^{4′};
R¹ is selected from:
(a) C₁₋₆-alkyl,
(b) C₁₋₆-alkoxy-C₁₋₆-alkyl,
(c) C₃₋₆-alkenyl,
(d) hydroxy-C₁₋₆-alkyl,
(e) halo-C₁₋₆-alkyl,
(f) aryl,
(g) arylcarbonylmethyl,
(h) aryl-C₂₋₆-alkenyl,
(i) aryl-C₁₋₆-alkyl,
(j) C₃₋₇-cycloalkyl,
(k) heteroaryl,
(l) 4-piperidinyl,
(m) N-substituted 4-piperidinyl, wherein the substituents are selected from C₁₋₆-alkyl, aryl, heteroaryl, aryl-C₁₋₆-alkyl and heteroaryl-C₁₋₆-alkyl,
(n) heteroaryl-C₁₋₆-alkyl,
wherein any heteroaryl or aryl residue, alone or as part of another group, is optionally substituted, independently, in one or more positions with substituents having the values as defined for R^{m} and R^{m′};
R^{m} and R^{m′} are each independently selected from:
(a) hydrogen,
(b) halogen,
(c) C₁₋₆-alkyl,
(d) hydroxy,
(e) C₁₋₆-alkoxy,
(f) C₂₋₆-alkenyl,
(g) phenyl,
(h) phenoxy,
(i) benzyloxy,
(j)benzoyl,
(k) —OCF₃,
(l) —CN,
(m) hydroxy-C₁₋₆-alkyl,
(n) halo-C₁₋₆-alkyl,
(o) —NR¹⁰R¹⁰,
(p) —NO₂,
(q) —CONR¹⁰R¹⁰,
(r) —NHSO₂R¹¹,
(s) —NR⁸COR¹¹,
(t) —SO₂NR⁸R¹¹,
(u) —C(═O)R¹¹,
(v) C₁₋₆-alkoxycarbonyl,
(w) —S(O)ₑR¹¹, wherein e is 0, 1, 2 or 3,
(x) —SCF₃,
(y) —CHF═CH₂,
(aa) —OCF₂H, or
(ab) ethynyl;
and with the proviso that, R^{m,} is attached to a carbon atom in ring B;
and with the further proviso that when one of W₁, W₂ and W₃ in Formula (I) is a nitrogen atom and both [symbol] represent single bonds the said nitrogen atom is attached to R^{m}, wherein R^{m} is selected from hydrogen or C₁₋₄-alkyl and v is 1;
and with the further proviso that when W₁, W₂ and W₃ in Formula (I) are each a carbon
atom and both [symbol] represent single bonds, R^{m} is selected from hydrogen or methyl; and with the further proviso that when R^{m} or R^{m′}, as substituents on ring A and B in Formula (I), are selected from phenyl, phenoxy, benzyloxy and benzoyl, the phenyl or aryl ring thereof may be optionally substituted by C₁₋₄-alkyl, halogen, C₁₋₄-alkoxy, C₁₋₄-alkylthio, trifluoromethyl, hydroxymethyl or cyano;
wherein R^{m} and R⁴ may be linked to each other to form a fused substituent of Formula (IV) provided that R^{m} is attached to W₁: 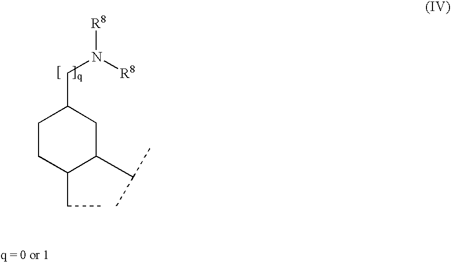
when U is CR⁴ or CHR⁴, R⁴ is a group selected from: 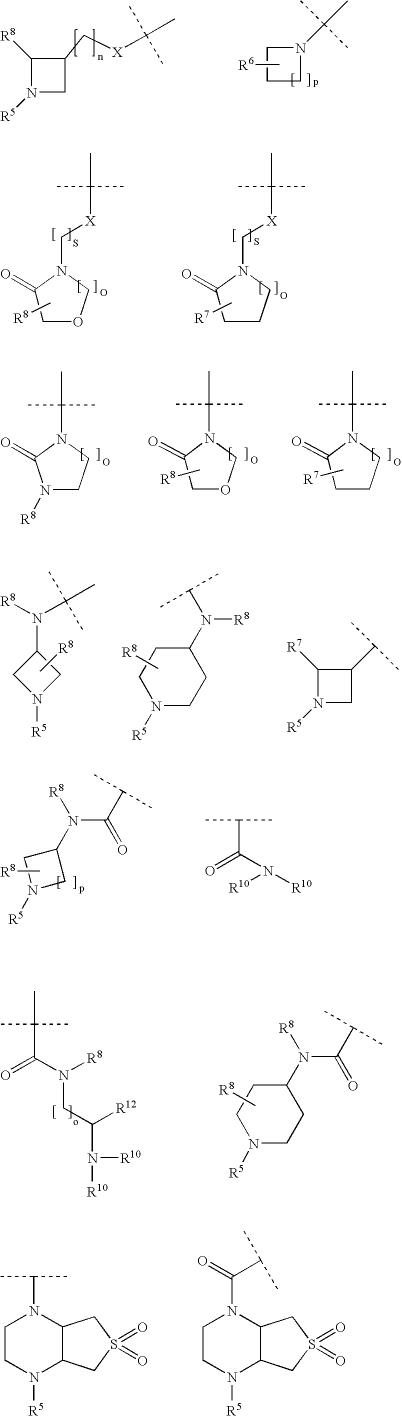 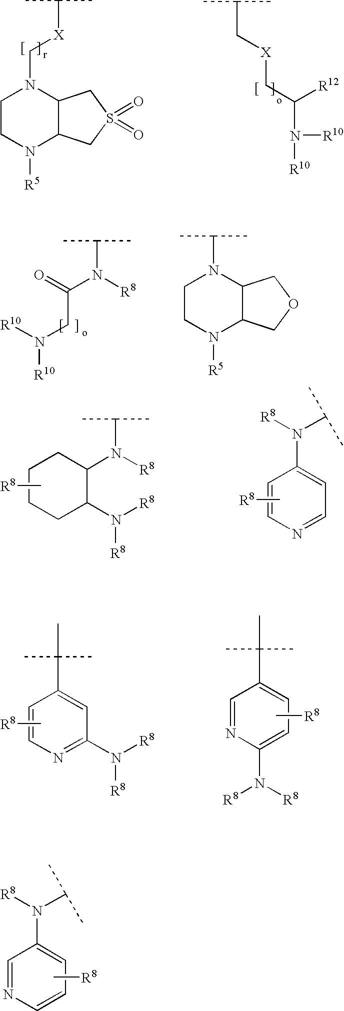
wherein:

n=0, 1, or 2,

o=1 or 2,

p=1, 2, 3, or 4,

r=2 or 3,

s=1, 2 or 3;
when U is CHR⁴, R⁴ is additionally selected from the following groups: 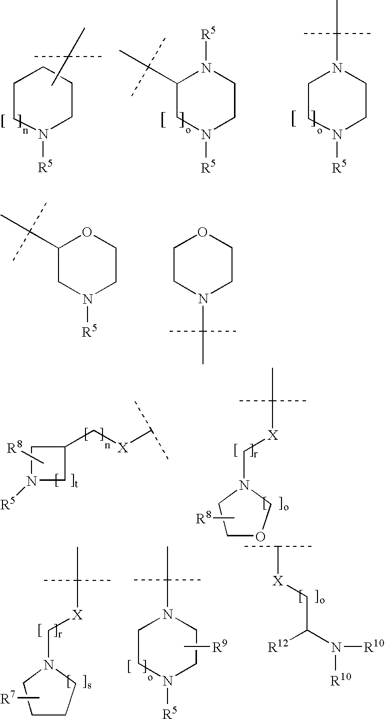
wherein:

n=0, 1 or 2,

o=1 or 2,

t=2, 3 or 4,

r=2 or 3,

s=1, 2 or 3;

wherein X is selected from O, NR⁸ and S;

wherein R⁵ is independently a group selected from:

(a) hydrogen,

(b) C₁₋₆-alkyl,

(c) 2-cyanoethyl,

(d) hydroxy-C₂₋₆-alkyl,

(e) C₃₋₆-alkenyl,

(f) C₃₋₆-alkynyl,

(g) C₃₋₇-cycloalkyl,

(h) C₃₋₇-cycloalkyl-C₁₋₄-alkyl,

(i) C₁₋₆-alkoxy-C₂₋₆-alkyl,

(j) aryl-C₁₋₆-alkyl,

(k) heteroaryl-C₁₋₆-alkyl,

(l) 3,3,3-trifluoropropyl,

wherein any aryl and heteroaryl residue may be optionally

substituted with C₁₋₄-alkyl, halogen, C₁₋₄-alkoxy, C₁₋₄-alkylthio, trifluoromethyl or cyano;

R⁶ is selected from:

(a) hydrogen,

(b) C₁₋₄-alkyl,

(c) hydroxy-C₁₋₄-alkyl,

(d) C₁₋₄-alkoxy-C₁₋₄-alkyl,

(e) halo-C₁₋₄-alkyl,

(f) —NR⁸R⁸, provided that the said —NR⁸R⁸ group is not attached to a carbon atom adjacent to a ring nitrogen atom,

(g) —CO—NR⁸R⁸;

(h) hydroxy, provided that the said hydroxy group is not attached to a carbon atom adjacent to a ring nitrogen atom;

R⁷ is selected from:

(a) hydrogen,

(b) C₁₋₄-alkyl,

(c) hydroxy-C₁₋₄-alkyl, or

(d) C₁₋₄-alkoxy-C₁₋₄-alkyl,

(e) hydroxy, provided that the said hydroxy group is not attached to a carbon atom adjacent to a heterocyclic ring nitrogen atom and that the said hydroxy group is attached to a heterocyclic ring not substituted with oxo;

R⁸ is each independently selected from:

(a) hydrogen, or

(b) C₁₋₆-alkyl,

R⁹ is selected from:

(a) hydrogen,

(b) C₁₋₄-alkyl, wherein one or two groups may be present at any carbon atom, or when two groups are present at the same carbon atom they may together form a cyclopropane ring,

(c) hydroxy-C₁₋₄-alkyl,

(d) C₁₋₄-alkoxy-C₁₋₄-alkyl,

(e) halo-C₁₋₄-alkyl,

R¹⁰ is each independently selected from:

(a) hydrogen,

(b) C₁₋₆-alkyl,

(c) hydroxy-C₂₋₄-alkyl,

(d) C₃₋₇-cycloalkyl, or

(e) C₁₋₄-alkoxy-C₂₋₄-alkyl,

wherein the two R¹⁰ groups together with the nitrogen to which they are attached form a heterocyclic ring; and when the two R¹⁰ groups form a piperazine ring, the nitrogen of the piperazine ring that allows the substitution may be substituted with a group selected from R⁵;

R¹¹ is selected from:

(a) C₁₋₆-alkyl,

(b) aryl, or

(c) heteroaryl,

wherein aryl and heteroaryl may be optionally substituted with C₁₋₄-alkyl, halogen, C₁₋₄-alkoxy, C₁₋₄-alkylthio, trifluoromethyl, hydroxymethyl or cyano;

R¹² is selected from:

(a) hydrogen, or

(b) methyl;

when U is R⁴R^{4′}, R⁴ and R^{4′} are linked to each other to form a heterocyclic ring selected from pyrrolidine or piperidine, wherein the N atom may be substituted by a group selected from R⁵; and pharmaceutically acceptable salts, hydrates, solvates, geometrical isomers, tautomers, optical isomers, and prodrug forms thereof.

It is preferred in Formula (I) that:

P is selected from 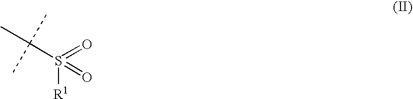
each of W₁, W₂, W₃, Z and Y is a carbon atom provided that both [symbol] in Formula (I) represent double bonds; or
one of W₁, W₂, W₃, Z and Y is a nitrogen atom, while the remainder being carbon atoms, provided that both [symbol] in Formula (I) represent single bonds;
U is selected from CHR⁴, CR⁴ and CR⁴R^{4′}, provided that when the dotted line connecting W₁ and U is a double bond, then U is CR⁴; and further provided that when the dotted line connecting W₁ and U is a single bond, then U is selected from CHR⁴ and CR⁴R^{4′};
R¹ is selected from:
(f) aryl,
(h) aryl-C₂₋₆-alkenyl,
(i) aryl-C₁₋₆-alkyl,
(j) C₃₋₇-cycloalkyl,
(k) heteroaryl,
(n) heteroaryl-C₁₋₆-alkyl,
wherein any heteroaryl or aryl residue, alone or as part of another group, is optionally substituted, independently, in one or more positions with substituents having the values as defined for R^{m} and R^{m′};
R^{m} and R^{m′} are each independently selected from:
(a) hydrogen,
(b) halogen,
(c) C₁₋₆-alkyl,
(d) hydroxy,
(e) C₁₋₆-alkoxy,
(f) C₂₋₆-alkenyl,
(k) —OCF₃,
(l) —CN,
(m) hydroxy-C₁₋₆-alkyl,
(n) halo-C₁₋₆-alkyl,
(o) —NR¹⁰R¹⁰,
(q) —CONR¹⁰R¹⁰,
(r) —NHSO₂R¹¹,
(s) —NR⁸COR¹¹,
(t) —SO₂NR⁸R¹¹,
(u) —C(═O)R¹¹,
(w) —S(O)ₑR¹¹, wherein e is 0, 1, 2 or 3,
(x) —SCF₃,
(y) —CHF═CH₂,
(aa) —OCF₂H, or
(ab) ethynyl;
and with the proviso that, R^{m,} is attached to a carbon atom in ring B; and
with the further proviso that when one of W₁, W₂ and W₃ in Formula (I) is a nitrogen atom and both [symbol] represent single bonds the said nitrogen atom is attached to R^{m}, wherein R^{m} is selected from hydrogen or C₁₋₄-alkyl and v is 1; and
with the further proviso that when W₁, W₂ and W₃ in Formula (I) are each a carbon atom and both [symbol] represent single bonds, R^{m} is selected from hydrogen or methyl; and with the further proviso that when R^{m} and R^{m,} are substituents on ring A and B, R^{m} and R^{m,} are independently selected from: hydrogen, halogen, methyl, methoxy, trifluoromethyl, hydroxymethyl or cyano;

when U is CR⁴ or CHR⁴, R⁴ is a group selected from: 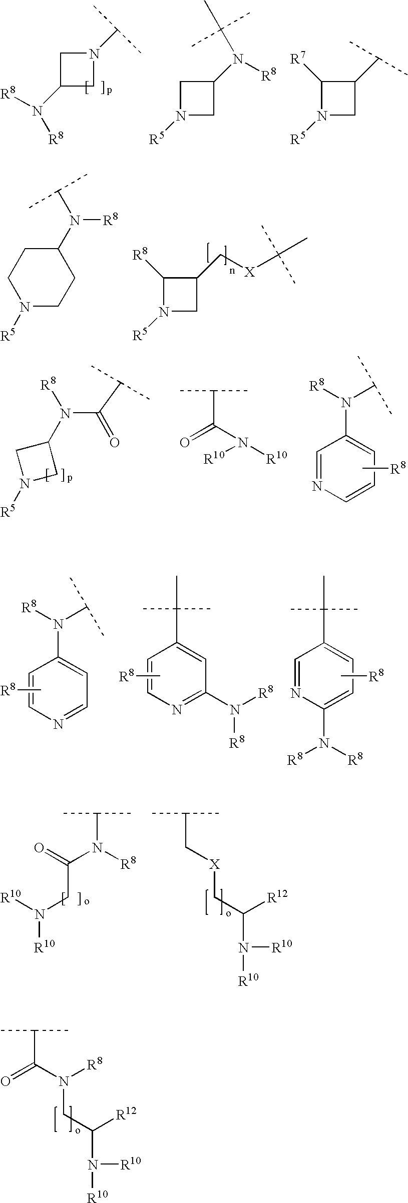
wherein:

n=0, 1, or 2,

o=1 or 2

p=1, 2, 3, or 4,
when U is CHR⁴,R⁴ is additionally selected from the following groups: 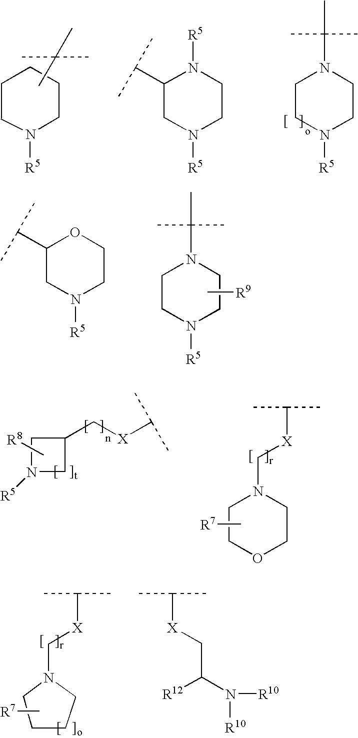
wherein:

n=0, 1 or 2,

o=1 or 2,

t=2, 3 or 4,

r=2 or 3,

wherein X is selected from O and NR⁸;

wherein R⁵ is independently a group selected from:

(a) hydrogen,

(b) C₁₋₆-alkyl,

(c) 2-cyanoethyl,

(d) hydroxy-C₂₋₄-alkyl,

(e) C₃₋₆-alkenyl,

(h) C₃₋₇-cycloalkyl-C₁₋₄-alkyl, or

(i) C₁₋₄-alkoxy-C₂₋₄-alkyl,

R⁷ is selected from:

(a) hydrogen,

(b) C₁₋₄-alkyl,

(c) hydroxy-C₁₋₂-alkyl, or

(d) C₁₋₂-alkoxy-C₁₋₂-alkyl;

R⁸ is each independently selected from:

(a) hydrogen, or

(b) C₁₋₆-alkyl,

R⁹ is selected from:

(a) hydrogen,

(b) C₁₋₄-alkyl, wherein one or two groups may be present at any carbon atom, or when two groups are present at the same carbon atom they may together form a cyclopropane ring,

(c) hydroxy-C₁₋₂-alkyl,

(d) C₁₋₂-alkoxy-C₁₋₂-alkyl,

(e) halo-C₁₋₂-alkyl,

R¹⁰ is each independently selected from:

(a) hydrogen,

(b) C₁₋₄-alkyl,

(c) hydroxy-C₂₋₄-alkyl

wherein the two R¹⁰ groups together with the nitrogen to which they are attached form a heterocyclic ring; and when the two R¹⁰ groups form a piperazine ring, the nitrogen of the piperazine ring that allows the substitution may be substituted with a group selected from R⁵;

R¹¹ is selected from:

(a) C₁₋₄-alkyl

R¹² is selected from:

(a) hydrogen, or

(b) methyl;

when U is R⁴R^{4′}, R⁴ and R^{4′} are linked to each other to form a heterocyclic ring selected from pyrrolidine or piperidine, wherein the N atom may be substituted by a group R⁵ selected from:

(a) hydrogen,

(b) C₁₋₄-alkyl,

(d) hydroxy-C₂₋₄-alkyl,

(i) C₁₋₄-alkoxy-C₂₋₄-alkyl,

(k) 2-cyanoethyl.

Preferred compounds are:

4′-Methyl-1′-(2-naphthylsulphonyl)-1′,4′,5′,6′-tetrahydrospiro{piperidine-2,7′-pyrrolo[3,2-b]pyridine} hydrochloride,
4′-Methyl-1′-(4-bromophenylsulphonyl)-1′,4′,5′,6′-tetrahydrospiro{piperidine-2,7′-pyrrolo[3,2-b]pyridine} hydrochloride,
4′-Methyl-1′-(5-bromo-2-thienylsulphonyl)-1′,4′,5′,6′-tetrahydrospiro{piperidine-2,7′-pyrrolo[3,2-b]pyridine} hydrochloride,
4′-Methyl-1′-(2-thienylsulphonyl)-1′,4′,5′,6′-tetrahydrospiro{piperidine-2,7′-pyrrolo[3,2-b]pyridine} hydrochloride,
N-(1-Benzenesulfonyl-1H-indol-4-yl)-2-(2-hydroxy-ethylamino)-acetamide,
1-Benzenesulfonyl-1H-indol-4-yl)-pyridin-4-yl-amine,
N-(4-piperazin-1-yl-1H-indol-1-yl)benzenesulfonamide hydrochloride, and
3-[(4-Methylphenyl)sulfonyl]-6,7,8,9-tetrahydro-3H-benzo[e]indol-8-amine trifluoroacetate

Another object of the present invention is a process for the preparation of a compound as mentioned above, comprising the following steps:
1) reaction of 2-(2-ethylamino)pyrrole and 1-methylpiperazine-4-one to give 4′-methyl-1′,4′,5′,6′-tetrahydrospiro{piperidine-2,7′-pyrrolo[3,2-b]pyridine}; and
2) reaction of the product from step a) with an arylsulphonyl chloride in the presence of a base.

Another object of the present invention is a process for the preparation of a compound as mentioned above, by reaction of 1-benzensulfonyl-1H-indol-4-ylamine and bromoacetyl bromide and further reaction with ethanolamine.

Another object of the present invention is a process for the preparation of a compound as mentioned above, by reductive amination of 3-(toluene-4-sulfonyl)-6,9-dihydro-3H, 7H-benzo[e]indol-8-one in the presence of sodium cyanoborohydride and ammonium acetate.

Another object of the present invention is a compound as mentioned above for use in therapy, especially for use in the treatment or prophylaxis of a 5-HT₆ receptor-related disorder, to achieve reduction of body weight and of body weight gain.

Another object of the present invention is a pharmaceutical formulation comprising a compound as mentioned above as active ingredient, in combination with a pharmaceutically acceptable diluent or carrier, especially for use in the treatment or prophylaxis of a 5-HT₆ receptor-related disorder, to achieve reduction of body weight and of body weight gain.

Another object of the present invention is a method for treating a human or animal subject suffering from a 5-HT₆ receptor-related disorder, to achieve reduction of body weight and of body weight gain. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the 5-HT₆ receptor-related disorder, to achieve reduction of body weight and of body weight gain. Identifying a subject in need of such treatment can be in the judgment of a subject or a health care professional and can be subjective (e.g., opinion) or objective (e.g., measurable by a test or diagnostic method).

Another object of the present invention is a method for the treatment or prophylaxis of a 5-HT₆ receptor-related disorder, to achieve reduction of body weight and of body weight gain, which comprises administering to a subject in need of such treatment an effective amount of a compound as mentioned above.

Another object of the present invention is a method for modulating 5-HT₆ receptor activity, which comprises administering to a subject in need of such treatment an effective amount of a compound as mentioned above.

Another object of the present invention is the use of a compound as mentioned above for the manufacture of a medicament for use in the prophylaxis or treatment of a 5-HT₆ receptor-related disorder, to achieve reduction of body weight and of body weight gain.

The compounds as mentioned above may be agonists, partial agonists or antagonists for the 5-HT₆ receptor. Preferably, the compounds act as partial agonists or antagonists for the 5-HT₆ receptor.

Examples of 5-HT₆ receptor-related disorders are obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder (ADHD), withdrawal from drug abuse, neurodegenerative diseases characterized by impaired neuronal growth, and pain.

The compounds and compositions are useful for treating diseases, to achieve reduction of body weight and of body weight gain. The diseases include obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder (ADHD), withdrawal from drug abuse, neurodegenerative diseases characterized by impaired neuronal growth, and pain. In one aspect, the invention relates to a method for treating or preventing an aforementioned disease comprising administering to a subject in need of such treatment an effective amount or composition delineated herein.

Another object of the present invention is a cosmetic composition comprising a compound as mentioned above as active ingredient, in combination with a cosmetically acceptable diluent or carrier, especially for use in the prophylaxis or treatment of a 5-HT₆ receptor-related disorder, to achieve reduction of body weight and of body weight gain.

Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted by one or more halogen atoms. Examples of said halo-C₁₋₆-alkyl include 2-fluoroethyl, fluoromethyl, trifluoromethyl and 2,2,2-trifluoroethyl. Likewise, “aryl-C₁₋₆-alkyl” means a C₁₋₆-alkyl group substituted by one or more aryl groups.

Unless otherwise stated or indicated, the term “hydroxy-C₁₋₆-alkyl” denotes a straight or branched alkyl group that has a hydrogen atom thereof replaced with OH. Examples of said hydroxy-C₁₋₆-alkyl include hydroxymethyl, 2-hydroxyethyl, 2-hydroxypropyl and 2-hydroxy-2-methylpropyl.

Unless otherwise stated or indicated, the term “C₁₋₆-alkoxy” denotes a straight or branched alkoxy group having from 1 to 6 carbon atoms. Examples of said C₁₋₆-alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term C₁₋₆-alkoxy-C₁₋₆-alkyl denotes a straight or branched alkoxy group having from 1 to 6 carbon atoms connected to an alkyl group having from 1 to 6 carbon atoms. Examples of said C₁₋₆-alkoxy-C₁₋₆-alkyl include methoxymethyl, ethoxymethyl, iso-propoxymethyl, n-butoxymethyl, t-butoxymethyl and straight- and branched-chain pentoxymethyl. For parts of the range “C₁₋₆-alkoxy-C₁₋₆-alkyl” all subgroups thereof are contemplated such as C₁₋₅-alkoxy-C₁₋₆-alkyl, C₁₋₄-alkoxy-C₁₋₆-alkyl, C₁₋₃-alkoxy-C₁₋₆-alkyl, C₁₋₂-alkoxy-C₁₋₆-alkyl, C₂₋₆-alkoxy-C₁₋₆-alkyl, C₂₋₅-alkoxy-C₁₋₆-alkyl, C₂₋₄-alkoxy-C₁₋₆-alkyl, C₂₋₃-alkoxy-C₁₋₆-alkyl, C₃₋₆-alkoxy-C₁₋₆-alkyl, C₄₋₅-alkoxy-C₁₋₆-alkyl, C₁₋₆-alkoxy-C₁₋₅-alkyl, C₁₋₆-alkoxy-C₁₋₄-alkyl, etc.

Unless otherwise stated or indicated, the term “C₂₋₆-alkenyl” denotes a straight or branched alkenyl group having from 2 to 6 carbon atoms. Examples of said C₂₋₆-alkenyl include vinyl, allyl, 2,3-dimethylallyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₄₋₅-alkenyl, etc. Likewise, “aryl-C₂₋₆-alkenyl” means a C₂₋₆-alkenyl group substituted by one or more aryl groups. Examples of said aryl-C₂₋₆-alkenyl include styryl and cinnamyl.

The term “oxo” denotes

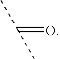

Unless otherwise stated or indicated, the term “C₃₋₆-alkynyl” denotes a straight or branched alkynyl group having from 3 to 6 carbon atoms. Examples of said C₃₋₆-alkynyl include 1-propynyl, 1-butynyl, and 1-hexynyl. For parts of the range “C₂₋₆-alkynyl” all subgroups thereof are contemplated such as C₃₋₅-alkynyl, C₃₋₄-alkynyl, C₃₋₆-alkynyl, C₄₋₅-alkynyl, etc.

Unless otherwise stated or indicated, the term “C₃₋₇-cycloalkyl” denotes a cyclic alkyl group having a ring size from 3 to 7 carbon atoms. Examples of said cycloalkyl include cyclopropyl, cyclobutyl, cyclopentyl, cyclohexyl, methylcyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc.

Unless otherwise stated or indicated, the term “aryl” refers to a hydrocarbon ring system having at least one aromatic ring. Examples of aryls are phenyl, pentalenyl, indenyl, indanyl, 1,2,3,4-tetrahydronaphthyl, 1-naphthyl, 2-naphthyl, fluorenyl and anthryl. The aryl rings may be optionally substituted. Likewise, phenoxy refers to a phenyl group bonded to an oxygen atom.

The term “heteroaryl” refers to a mono- or bicyclic aromatic ring system, only one ring need be aromatic, and the said heteroaryl moiety can be linked to the remainder of the molecule via a carbon or nitrogen atom in any ring, and having from 5 to 10 ring atoms (mono- or bicyclic), in which one or more of the ring atoms are other than carbon, such as nitrogen, sulphur, oxygen and selenium. Examples of such heteroaryl rings include furyl, pyrrolyl, thienyl, oxazolyl, isoxazolyl, imidazolyl, thiazolyl, isothiazolyl, pyridinyl, pyrimidinyl, pyrazinyl, chromanyl, quinazolinyl, indolyl, isoindolyl, indolinyl, isoindolinyl, indazolyl, pyrazolyl, pyridazinyl, quinolinyl, isoquinolinyl, benzofuranyl, dihydrobenzofuranyl, benzodioxolyl, benzodioxinyl, benzothienyl, benzimidazolyl, benzothiazolyl, benzothiadiazolyl, and benzotriazolyl groups. If a bicyclic heteroaryl ring is substituted, it may be substituted in any ring.

Unless otherwise stated or indicated, the term “heterocyclic” refers to a non-aromatic (i.e., partially or fully saturated) mono- or bicyclic ring system having 4 to 10 ring atoms with at least one heteroatom such as O, N, or S, and the remaining ring atoms are carbon. Examples of heterocyclic groups include piperidyl, tetrahydropyranyl, tetrahydrofuranyl, azepinyl, azetidinyl, pyrrolidinyl, morpholinyl, imidazolinyl, thiomorpholinyl, pyranyl, dioxanyl, and piperazinyl groups. When present, the sulfur atom may optionally be in an oxidized form (i.e., S═O or O═S═O). Examples of heterocyclic groups containing sulfur in oxidized form include octahydrothieno[3,4b]pyrazine 6,6-dioxide and thiomorpholine 1,1-dioxide.

Unless otherwise stated or indicated, the term “halogen” shall mean fluorine, chlorine, bromine or iodine.

The term —S(O)_{c}R¹¹, wherein e is 0, 1, 2 or 3, has the meaning as illustrated by

Formula (V)-(VIII):

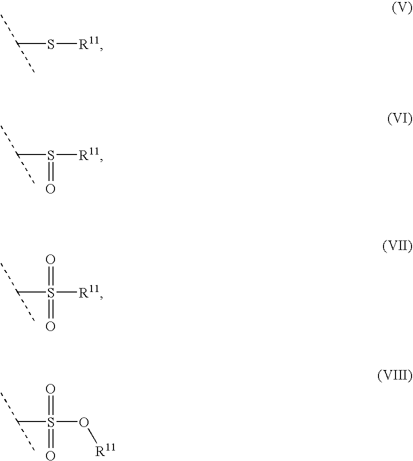

The term “leaving group” refers to a group to be displaced from a molecule during a nucleophilic displacement reaction. Examples of leaving groups are iodide, bromide, chloride, methanesulphonate, hydroxy, methoxy, thiomethoxy, tosyl, or suitable protonated forms thereof (e.g., H₂O, MeOH), especially bromide and methanesulphonate.

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15; and “The Organic Chemistry of Drug Design and Drug Action” by Richard B. Silverman. Chapter 8, p 352. (Academic Press, Inc. 1992. ISBN 0-12-643730-0).

The following abbreviations have been used:

CV means Coefficient of Variation,

DMSO means dimethyl sulphoxide,

EDTA means ethylenediamine tetraacetic acid,

EGTA means ethylenebis(oxyethylenenitrilo)tetraacetic acid,

HEPES means 4-(2-hydroxyethyl)-1-piperazineethanesulfonic acid,

HPLC means high performance liquid chromatography,

LSD means lysergic acid, diethylamide,

MeCN means acetonitrile,

SPA means Scintillation Proximity Assay,

t-BuOK means potassium tert-butoxide, and

THF means tetrahydrofuran.

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z-double bond isomer forms. All isomeric forms are contemplated.

The compounds of the Formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parentral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to-obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

TABLE 1

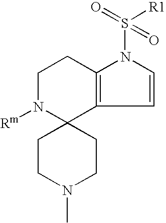

| EXAMPLE | R¹ | R^{m} |
|---|---|---|
| 1 | 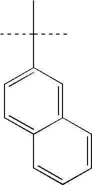 | H |
| 2 | 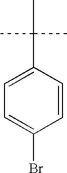 | H |
| 3 | 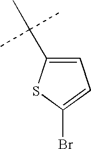 | H |
| 4 | 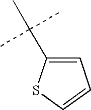 | H |

Methods

¹H nuclear magnetic resonance (NMR) and ¹³C NMR were recorded on a Bruker Advance DPX 400 spectrometer at 400.1 and 100.6 MHz, respectively. All spectra were recorded using residual solvent or tetramethylsilane (TMS) as internal standard. Infra red (IR) spectra were recorded on a Perkin-Elmer Spectrum 1000 FT-IR spectrophotometer. Ionspray mass spectrometry (MS) spectra were obtained on a Perkin-Elmer API 150EX mass spectrometer. Accurate mass measurements were performed on a Micromass LCT dual probe. Preparative HPLC/MS was performed on a Waters/Micromass Platform ZQ system equipped with System A: ACE 5 C8 column (19×50 mm), eluents: MilliQ water, MeCN and MilliQ/MeCN/0.1% TFA and system B: Xterra MS C18, 5 μm column (19×50 mm), eluents: MilliQ water, MeCN and NH₄HCO₃ (100 mM). Analytical HPLC were performed on Agilent 1100, column: ACE 3 C8 (system A) or column: YMC-Pack (system B), eluents: MilliQ/0.1% TFA and MeCN. Elemental analyses were performed on a Vario El instrument. Preparative flash chromatography was performed on Merck silica gel 60 (230-400 mesh).

### EXPERIMENTAL

### Synthesis of 4′-methyl-1′,4′,5′,6′-tetrahydrospiro{piperidine-2,7′-pyrrolo[3,2-b]pyridine}

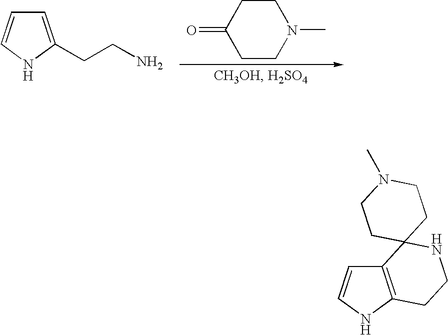

A mixture of 2-(2-ethylamino)pyrrole (Herz W. J. Am. Chem. Soc. 75, 483, 1953; Wasley J. W. F, EP 338989 B, 1989) (9.6 g, 87 mmol) and 1-methylpiperazin-4-one (9.85 g, 87 mmol) in benzene (100 mL) was completely evaporated under vacuo. The dry residue was dissolved in methanol (50 mL). H₂SO₄ (43 mL) in methanol (107 mL) was added to the methanol solution at 0° C. (ice). The cooling bath was removed and the mixture was stirred for 2 hours at room temperature. It resulted in the appearance of a white dense precipitate. Then the mixture was held overnight at −20° C. The precipitate was filtered and dissolved in minimal amount of NaOH in water (30%). The organic material was extracted with ethyl acetate (100 mL×4). The organic phases were combined and dried by K₂CO₃ followed by filtration. The volatiles were eliminated under vacuo. The residue was triturated with cold ethyl acetate, filtered and washed by minimal amount of cold ethyl acetate to yield 7.6 g (44%) of product.

### General method for the synthesis of 4′-methyl-1′,4′,5′,6′-tetrahydrospiro{piperidine-2,7′-pyrrolo[3,2-b]pyridine}sulphonamides

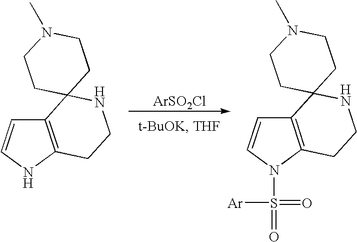

A suspension of t-BuOK (160 mg, 1.4 mmol) in anhydrous THF (3 mL) was added to the mixture of 4′-methyl-1′,4′,5′,6′-tetrahydrospiro{piperidine-2,7′-pyrrol[3,2-b]pyridine} (205 mg, 1 mmol) in THF. The mixture was heated under stirring (complete dissolution was observed at 40° C.) followed by the addition of the corresponding sulfonyl chloride (1.2 mmol) in THF (3 mL). The mixture was papidly heated to the boiling point and then cooled. The reaction mixture was poured into water, extracted with ethyl acetate, dried (K₂CO₃) and evaporated. The product was isolated by column chromatography on silica gel (CHCl₃/MeOH:10/1). The yields of compounds synthesized by this method vary from 18 to 62%.

### Example 1

### 4′-Methyl-1′-(2-naphthylsulphonyl)-1′,4′,5′,6′-tetrahydrospiro{piperidine-2,7′-pyrrolo[3,2-b]pyridine} hydrochloride

28.3 mg of material was isolated. ¹H NMR (270 MHz, Methanol-d₄) δ 2.25-2.50 (m, 4H), 2.96 (s, 3H), 3.19 (app. t., 2H), 3.52-5.65 (m, 6H), 7.56 (d, 1H, J=3.22 Hz), 7.72 (dq, 1H, J=1.48 Hz), 7.83 (dd, 1H, J=1.86 Hz, J=8.98 Hz), 7.99-8.02 (m, 1H), 8.09 (d, 2H, J=8.98 Hz), 8.11-8.15 (m, 1H), 8.67 (d, 1H, J=1.73 Hz). HPLC purity 96%.

### Example 2

### 4′-Methyl-1′-(4-bromophenylsulphonyl)-1′,4′,5′,6′-tetrahydrospiro{piperidine-2,7′-pyrrolo[3,2-b]pyridine} hydrochloride

29.8 mg of material was isolated. ¹H NMR (270 MHz, DMSO-d₆) δ 1.45-1.50 (bd, 2H) Hz), 1.71-1.83 (dt, 2H, J=4.21 Hz), 2.15 (s, 3H), 2.36 (app.t, 2H), 2.45-2.55 (m, 2H), 2.59 (app.t, 2H), 2.88 (app.t, 2H), 2.86 (app.t, 2H), 6.32 (d, 1H, J=3.46 Hz), 7.15 (d, 1H, J=3.46 Hz), 7.43 (d, 1H, J=3.96 Hz), 7.71 (d, 1H, J=3.96 Hz). HPLC purity 95%.

### Example 3

### 4′-Methyl-1′-(5-bromo-2-thienylsulphonyl)-1′,4′,5′,6′-tetrahydrospiro{piperidine-2,7′-pyrrolo[3,2-b]pyridine} hydrochloride

254 mg of material was isolated. ¹H NMR (270 MHz, DMSO-d₆) δ 1.40-1.44 (bd, 2H, J=12.62 Hz), 1.72 (dt, 2H, J=4.21 Hz), 2.22 (s, 3H), 2.25 (bt, 2H, J=10.89 Hz), 2.45-2.55 (m, 2H), 2.42-2.46 (m, 2H), 2.83 (app.t, 2H), 2.86 (app.t, 2H), 6.29 (d, 1H, J=3.46 Hz), 7.15 (d, 1H, J=3.22 Hz), 7.23 (dd, 1H, J=4.95 Hz, J=3.96 Hz), 7.83 (dd, 1H, J=3.84 Hz, J=1.36 Hz), 8.10 (dd, 1H, J=4.95 Hz, J=1.24 Hz). HPLC purity 95%.

### Example 4

### 4′-Methyl-1′-(2-thienylsulphonyl)-1′,4′,5′,6′-tetrahydrospiro{piperidine-2,7′-pyrrolo[3,2-b]pyridine} hydrochloride

67.6 mg of material was isolated. ¹H NMR (270 MHz, Methanol-d₄) δ 1.45 (d, 2H J=13.11 Hz), 1.75 (dt, 2H, J=3.96 Hz), 2.18 (s, 3H), 2.25-2.40 (m, 2H), 2.40-2.65 (m, 4H), 2.59 (app. t, 2H), 2,86 (app.t, 2H), 6.29 (d, 1H, J=3.46 Hz), 7.15 (d, 1H, J=3.22 Hz), 7.23 (dd, 1H, J=4.95 Hz, J=3.96 Hz), 7.83 (dd, 1H, J=3.84 Hz, J=1.36 Hz), 8.10 (dd, 1H, J=4.95 Hz, J=1.24 Hz). HPLC purity 95%.

TABLE 2

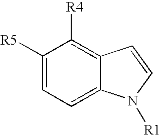

| Example | Chemical Name | R¹ | R⁴ | R⁵ |
|---|---|---|---|---|
| 5 | N-(1-Benzenesulfonyl-1H-indol-4-yl)-2-(2-hy- droxy-ethylamino)-acetamide | 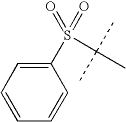 | 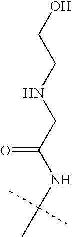 | H |
| 6 | 1-Benzenesulfonyl-1H-indol-4-yl)-py- ridin-4-yl-amine. | 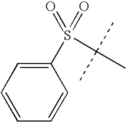 | 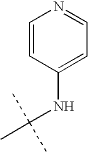 | H |
| 7 | N-(4-Piperazin-1-yl-1H-indol-1-yl)benzene- sulfonamide hydrochloride | 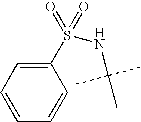 | 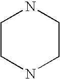 | H |
| 8 | 3-[(4-Methylphenyl)sulfonyl]-6,7,8,9-tetra- hydro-3H-benzo[e]indol-8-amine trifluoroacetate | 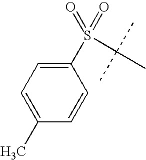 | 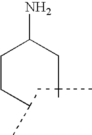 | |
| 9 | N′-(1-Benzenesulfonyl-1H-indol-4-yl- methyl)-N,N-dimethyl-ethane-1,2-diamine trifluoroacetate | 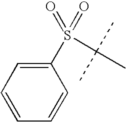 | 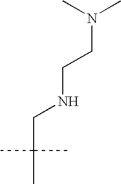 | |

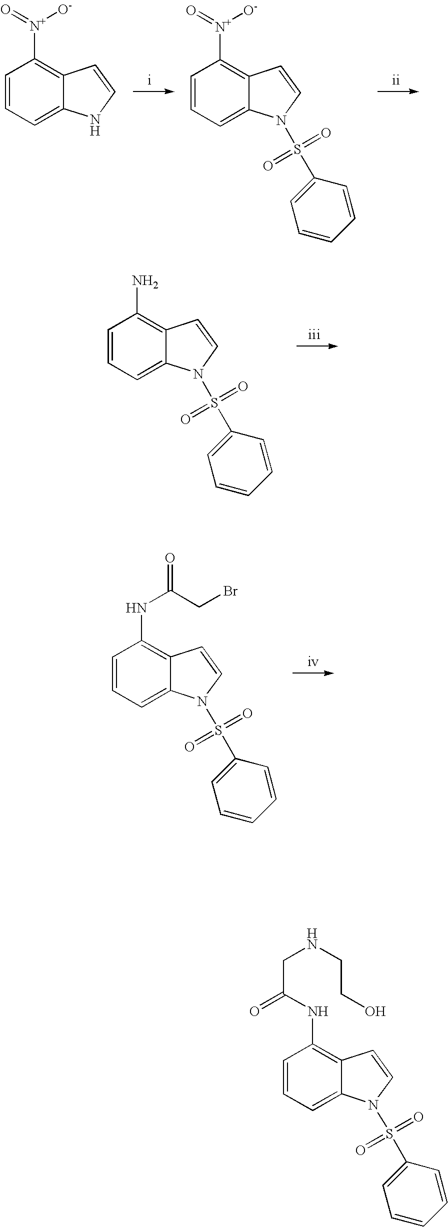 Legend to Scheme 1: i) N,N-diisopropylethylamine, acetonitrile, heat; ii) H₂, Pd/C methanol, ammonium formate, room temperature; iii) bromoacetyl bromide, NaHCO₃, CH₂Cl₂; iv) ethanolamine, KI, ethanol, heat.
Intermediate 1

### 4-Nitro-1 H-indole

p-Toluenesulfonic acid monohydrate (0.10 kg) was added to triethyl orthoformate (8.00 kg) at 111° C. The mixture was stirred for 5 min and then 3-nitro-o-toluidine (4.10 kg) was added portion wise. The addition is slightly exothermic and the speed of addition was adjusted to avoid letting the temperature fall below 111° C. The addition time was 65 min. During the addition, formed ethanol starts to distill off. After the addition was completed, the reaction mixture was distilled at atmospheric pressure for 70 min at 125° C. In total 4.5 L of ethanol was distilled off. When the distillation speed slowed down, vacuum (800 mbar-140 approx. 6L remained in the reactor. The reaction mixture was diluted with N,N-dimethylformamide (DMF) (15 L) and cooled to 43° C. Diethyl oxalate (4.20 kg) was added. To the resulting mixture, potassium tert-butoxide (4.08 kg) was added portionwise while keeping the temperature between 38-45° C. The addition time was 55 min. After completed addition, the reaction was stirred at 40-45° C. for 10 min. The reaction mixture was then added to water (90 L) at 50-60° C. The product crystallized during the addition. After cooling to 20° C., the product was isolated on a nutsche filter. The resulting product cake was washed with 25 L of water and dried at 80° C. while applying full vacuum. The product weighed 2.90 kg (66%) after drying. ¹H NMR (400 MHz, DMSO-d₆) δ ppm 7.05 (d, J=2.93 Hz, 1H) 7.28 (t, J=7.93 Hz, 1H) 7.75 (d, J=2.93 Hz, 1H) 7.89 (d, J=7.32 Hz, 1H) 8.04 (d, J=8.06 Hz, 1H) 11.96 (s, 1H). ¹³C NMR (101 MHz, DMSO-d₆) δ ppm 101.11 (s, 1 C) 116.79 (s, 1 C) 119.13 (s, 1 C) 120.06 (s, 1 C) 121.26 (s, 1 C) 130.65 (s, 1 C) 138.13 (s, 1 C) 139.09 (s, 1 C).

Intermediate 2

### 1-Benzenesulfonyl-4-nitro-1H-indole

A solution of 4-nitro-1H-indole (9.49 kg) in acetonitrile (69.5 kg) was heated to 81° C. N,N-Diisopropylethylamine (Hünigs base) (8.98 kg) was added followed by a portion wise addition of benzenesulfonyl chloride. The exotermic addition of benzenesulfonyl chloride was done with such speed that the temperature was kept between 75-81° C. The addition time was 45 min. after stirring the reaction mixture for 30 min at 80° C., analysis showed the disappearance of the starting material. Water (8.2 L) was added to the reaction mixture at 80° C. (NOTE! A serious error was made, as the recipe called for the addition of 16 L of water). The reaction mixture was kept at 80° C. for 50 min. Upon cooling, the product started to crystallize at approximately 74° C. The resulting slurry was cooled to 10° C. The product was isolated on a nutsche filter and washed with a mixture of acetonitrile (21.9 kg) and water (9.1 L). Drying at 70° C. and full vacuum gave 12.71 kg (72%) of the title compound. ¹H NMR (400 MHz, DMSO-d₆) δ ppm 7.34 (d, J=3.91 Hz, 1H) 7.55-7.65 (m, 3H) 7.69-7.76 (m, 1H) 8.05-8.09 (m, 2H) 8.18-8.23 (m, 2H) 8.42 (dd, J=8.30, 0.73 Hz, 1H). ¹³C NMR (101 MHz, DMSO-d₆) δ ppm 107.64 (s, 1 C) 119.75 (s, 1 C) 120.18 (s, 1 C) 124.20 (s, 1 C) 124.66 (s, 1 C) 126.78 (s, 2 C) 129.94 (s, 2 C) 131.17 (s, 1 C) 135.07 (s, 1 C) 135.36 (s, 1 C) 136.34 (s, 1 C) 139.96 (s, 1 C).

Intermediate 3

### 1-Benzenesulfonyl-1H-indol-4-ylamine

To a solution of 4-nitro-1H-indole (1 g, 3.3 mol) in MeOH (7 mL) under argon Pd/C (150 mg) and ammonium formate (3 g, 47 mmol) were added. The obtained mixture was heated to reflux temeprature for 1.5 h until complete disappearance of the starting nitro compound. The catalyst was filtered off, and the residue was washed with methanol. Then the solution was concentrated and purified by flash chromatography to give 0.72 g (80%) of the title compound. ¹H NMR (400 MHz, DMSO-d₆) δ ppm 5.57 (s, 2H) 6.39 (d, J=7.81 Hz, 1H) 6.95-7.05 (m, 2H) 7.12 (d, J=8.06 Hz, 1H) 7.50-7.58 (m, 3H) 7.59-7.67 (m, 1H) 7.91 (d, J=7.57 Hz, 2H). ¹³C NMR (101 MHz, DMSO-d₆) δ ppm 101.49 (s, 1 C) 107.29 (s, I C) 108.26 (s, 1 C) 118.55 (s, 1 C) 124.12 (s, 1 C) 126.61 (s, 1 C) 127.19 (s, 2 C) 130.25 (s, 2 C) 134.91 (s, 1 C) 136.11 (s, 1 C) 137.86 (s, 1 C) 142.91 (s, 1 C).

Intermediate 4

### N-(1-Benzenesulfonyl-1H-indol-4-yl)-2-bromo-acetamide

The solution of 1-benzenesulfonyl-1H-indol-4-ylamine (0.6 g, 2.2 mmol) in CH₂Cl₂ (10 mL) the solution of NaHCO₃ (0.84 g, 10 mmol) in water (10 mL) was added dropwise. Then bromoacetyl bromide (0.21 mL, 2.4 mmol) was added to the resulting mixture with simultaneous stirring. The reaction mixture was stirred for 30 min, and then the organic layer was separated and concentrated. The yield of product was 0.78 g (90%). The formation of the product was monitored by TLC (Thin Layer Chromatography). The compound was taken to the next step without further analysis.

### Example 5

### N-(1-Benzenesulfonyl-1H-indol-4-yl)-2-(2-hydroxy-ethylamino)-acetamide

To N-(1-benzenesulfonyl-1H-indol-4-yl)-2-bromo-acetamide (0.78 g, 2 mmol) dissolved in EtOH (5 mL) KI (0.07 g, 0.4 mmol) and ethanolamine (0.6 mL, 10 mmol) were added. The reaction mixture was heated to reflux temperature for 10 min until the completion of the reaction as indicated by TLC. The product was purified by column chromatography (eluent system CHCl₃/CH₃OH 5:1). The yield of product was 0.52 g (70%).Yield; 0.52 g (70%) of material was isolated; HPLC purity 95%; ¹H NMR (400 MHz, DMSO-d₆) δ ppm 2.62 (t, J=5.40 Hz, 2H) 3.47 (q, J=5.44 Hz, 2H) 4.65 (t, J=5.27 Hz, 2H) 6.99 (d, J=3.51 Hz, 1 H) 7.29 (t, J=8.16 Hz, 1H) 7.59 (t, J=7.78 Hz, 2H) 7.69 (t, J=7.53 Hz, 2H) 7.78-7.85 (m, 2H) 7.94-8.00 (m, 2H); MS (posEI-DIP) m/z 374 (M+H).

### Example 6

### 1-Benzenesulfonyl-1H-indol-4-yl)-pyridin-4-yl-amine

To the solution of 1-benzenesulfonyl-1H-indol-4-ylamine (0.5 g, 1.8 mmol) in DMF (4 mL) 4-bromopyridine hydrochloride (0.36 g, 1.8 mmol) and KI (0.07 g, 0.40 mmol) were added. The reaction mixture was heated to reflux temperature for 2 h. The reaction was monitored by TLC. The organic layer was concentrated and final compound was purified by flash-chromatography (eluent: CHCl₃). The yield of product was 0.175 g (35%). HPLC purity 98%; ¹H NMR (270 MHz, DMSO-d₆) δ 6.77-6.87 (m, 1H), 6.96-7.10 (m, 2H), 7.23-7.32 (m, 1H), 7.36-7.50 (m, 1H), 7.55-7.78 (m, 4H), 7.82-7.93 (m, 2H), 7.98-8.01 (m, 2H), 8.18-8.30 (m, 2H), 10.45 (brs, 1H); MS (posEI-DIP) m/z 350 (M+H).

Intermediate 5

### tert-Butyl 4-(1-amino-1H-indol-4-yl)piperazine-1-carboxylate

To a solution of tert-butyl 4-(1H-indol-4-yl)piperazine-1-carboxylate (0.56 g, 1.9 mmol) (WO 02/32863) in DMF (30 mL) at 0° C. was added KOH (1.04 g, 18.6 mmol), followed by hydroxylamine-O-sulfonic acid (0.42 g, 3.7 mmol), added portionwise over 30 min. After stirring at ambient temperature for 1 h, the mixture was filtered, and the filtrate was poured into ice water (200 mL) and extracted with ethyl acetate (3×100 mL). The organic layer was washed with water and brine, dried (MgSO₄), and filtered, and the filtrate was concentrated under reduced pressure. The resultant oil was purified by column chromatography on silica using gradient elution CHCl₃→CHCl₃+5% MeOH→CHCl₃+10% MeOH as eluent to yield 0.536 g of the product. HPLC purity 91%; MS (posEI-DIP) m/z 317 (M+H). (Larry Davies et al. J. Med. Chem, 1996, 39, 582-587).

### Example 7

### N-(4-piperazin-1-yl-1H-indol-1-yl)benzenesulfonamide hydrochloride

To a suspension of of NaH (0.05 g, 2.0 mmol; 50% oil dispersion) in 5 mL DMF at 0° C., was added a solution of tert-butyl 4-(1-amino-1H-indol-4-yl)piperazine-1-carboxylate (0.54 g, 1.7 mmol) in DMF (5 mL). After warming to 50° C. for 30 min, the solution was cooled to 0° C., and a solution of benzenesulfonyl chloride 0.30 g, 1.7 mmol) in DMF (3 mL) was slowly added. The mixture was stirred at room temperature over night and then concentrated under reduced pressure. Column chromatography on silica using CHCl₃+5% MeOH as eluent gave a crude intermediate which was dissolved in MeOH and HCl in ether (1M) was added. The mixture was stirred over 16 hours at room temperature and then concentrated to give 0.223 g crude product. The crude product was purified by preparative HPLC, converted to its HCl-salt and then lyophilized to give 0.010 g of the pure product as a brown solid. The solid was dried under vacuo at 60° C. for 5 days to remove all the solvent. Yield; 10 mg of material was isolated; HPLC purity 95%; ¹H NMR (270 MHz, Methanol-d₄) δ ppm 3.37-3.53 (m, 8H) 6.46 (appd, J=3.46 Hz, 1H) 6.66 (appd, J=7.55 Hz, 1H) 6.70-6.75 (m, 1H) 6.79 (appd, J=8.16 Hz, 1H) 6.89-7.02 (m, 1H) 7.50 (appt, J=7.67 Hz, 2H) 7.61-7.77 (m, 3H); MS (posEI-DIP) m/z 357 (M+H). (Larry Davies et al. J. Med. Chem, 1996, 39, 582-587). (Ishibashi, Hiroyuki; Akamatsu, Susumu; Iriyama, Hiroko; Iceda, Masazumi. Convenient synthesis of 4-alkyl, alkenyl, and alkynyl substituted N-(phenylsulfonyl)indoles. Chemical & Pharmaceutical Bulletin (1994), 42(10), 2150-3. Ishibashi, Hiroyuki; Tabata, Takashi; Hanaoka, Kyoko; Iriyama, Hiroko; Akamatsu, Susumu; Ikeda, Masazumi. A new, general entry to 4-substituted indoles. Synthesis of (S)-(−)-pindolol and (±)-chuangxinmycin. Tetrahedron Letters (1993), 34(3), 489-92).

### Example 8

### 3-[(4-Methylphenyl)sulfonyl]-6,7,8,9-tetrahydro-3H-benzo[e]indol-8-amine trifluoroacetate

To a suspension of 3-(toluene-4-sulfonyl)-6,9-dihydro-3H,7H-benzo[e]indol-8-one (0.017 g, 0.1 mmol) in dry methanol (2 mL) at room temperature was added first ammonium acetate (0.0387 g, 0.5 mmol) and then after 2 mins, sodium cyanoborohydride (0.0157 g, 0.03 mmol). The mixture was heated to 70° C. After 16 h, the sample was allowed to cool and then was treated with conc. aq. HCl until pH 2 was achieved. The mixture was washed with diethyl ether (2×20 mL) and then the aqueous phase was treated with 5M aq. NaOH. The resulting suspension was extracted with diethyl ether (2×20 mL), washed with brine (1×10 mL) and dried over anhydrous magnesium sulfate. The solvent was removed under reduced pressure and purification by preparative HPLC gave the desired product as a white solid (0.0027 g, 15%).HPLC 100%, R_{T}=2.828 min (system A, 5-60% MeCN over 3 min); 100%, R_{T}=2.451 min (system B, 5-60% MeCN over 3 min); 1H NMR (270 MHz, METHANOL-D4) δ ppm 1.21-1.39 (m, 2H) 2.08-2.24 (m, 1H) 2.33 (s, 3H) 2.77-3.07 (m, 2H) 3.55 (d, J=1.48 Hz, 2H) 6.71 (d, J=3.71 Hz, 1H) 7.09 (d, J=8.41 Hz, 1H) 7.29 (d, J=8.16 Hz, 2H) 7.64 (d, J=3.46 Hz, 1H) 7.70-7.88 (m, 3H); MS (ESI+) for C₁₉H₂₀N₂O₂S m/z 341 (M+H). The preparation of 3-(toluene-4-sulfonyl)-6,9-dihydro-3H,7H-benzo[e]indol-8-one is descibed in J. Med. Chem. 1995, 38, 2217-30.

Intermediate 6

### 4-Methyl-1-(phenylsulfonyl)-1H-indole

The material was prepared according to the literature method. HPLC purity 99%; ¹H NMR (400 MHz, DMSO-d₆) δ ppm 2.41 (s, 3H) 6.88 (d, J=3.76 Hz, 1H) 7.04 (d, J=7.53 Hz, 1H) 7.18-7.26 (m, 1H) 7.57 (t, J=7.65 Hz, 2H) 7.67 (t, J=7.40 Hz, 1H) 7.72-7.81 (m, 2H) 7.92-7.98 (m, 2H); MS (ESI+) for C₁₅H₁₃NO₂S m/z 272 (M+H)⁺. (Chemical & Pharmaceutical Bulletin (1994), 42(10), 2150-3, Tetrahedron Letters (1993), 34(3), 489-92).

Intermediate 7

### 4-(Bromomethyl)-1-(phenylsulfonyl)-1H-in dole

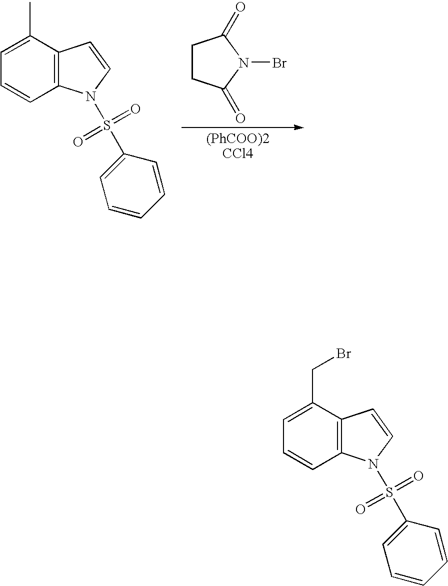

The compound was obtained using N-bromosuccinimide (1.2 equiv.), as bromination agent, and benzoyl peroxide (0.25 equiv.), as initiator, in CCl₄. The final product was purified by flash chromatography (using CCl₄ as eluent and obtained as white crystals (Yield: 3.5 g (61.6%); eluent-system chloroform-hexane 1:1). HPLC purity 92%; ¹H NMR (400 MHz, DMSO-d₆) δ ppm 4.94 (s, 2H) 7.04 (d, J=3.76 Hz, 1H) 7.28-7.37 (m, 2H) 7.59 (t, J=7.78 Hz, 2H) 7.69 (t, J=7.53 Hz, 1H) 7.89-7.94 (m, 2H) 8.00 (d, J=8.03 Hz, 2H); MS (ESI+) for C₁₅H₁₂BrNO₂S m/z 351 (M+H)⁺ (WO 9602502 A1 19960201).

### Example 9

### N′-(1-Benzenesulfonyl-1H-indol-4-ylmethyl)-N,N-dimethyl-ethane-1,2-diamine

The compound was prepared from Intermediate 7 and dimethylethylamine. Yield: 184 mg (98%); R_{T}=1.44 HPLC (System A. 10-97% MeCN over 3 min) and 99% R_{T}=1.31 (System B. 10-90% MeCN over 3 min). ¹H NMR (400 MHz, MeOD) δ ppm 2.93 (s, 6H) 3.44-3.61 (m, 4H) 4.51 (s, 2H) 7.05 (d, J=3.51 Hz, 1H) 7.42 (d, J=3.76 Hz, 2H) 7.50 (t, J=7.65 Hz, 2H) 7.61 (t, J=7.40 Hz, 1H) 7.80-7.85 (m, 1H) 7.96 (d, J=8.03 Hz, 2H) 8.09-8.15 (m, 1H), (ESI+) for C₁₉H₂₃N₃O₂S m/z 358 (M+H)+

Biological Tests

The ability of a compound according to the invention to bind to a 5-HT₆ receptor, and to be pharmaceutically useful, can be determined using in vivo and in vitro assays known in the art.

(a) 5-HT₆ Receptor Binding Assay

Binding affinity experiment for the human 5-HT₆ receptor are performed in HEK293 cells transfected with 5-HT₆ receptor using [³H]-LSD as labeled ligand according to the general method as described by Boess F. G et al. Neuropharmacology 36(4/5) 713-720, 1997.

Materials

Cell Culture

The HEK-293 cell line transfected with the human 5-HT₆ receptor was cultured in Dulbeccos Modified Eagles Medium containing 5% dialyzed foetal bovine serum, (Gibco BRL 10106-169), 0.5 mM sodium pyruvate and 400 μg/ml Geneticin (G-418) (Gibco BRL10131-019). The cells were passaged 1:10, twice a week.

Chemicals

The radioligand [³H] LSD 60-240 Ci/mmol, obtained from Amersham Pharmacia Biotech, (Buckinghamshire, England) was in ethanol and stored at −20° C. The compounds were dissolved in 100% DMSO and diluted with binding buffer.

Disposable

Compounds were diluted in Costar 96 well V-bottom polypropylene plates (Corning Inc. Costar, N.Y., USA). Samples were incubated in Packard Optiplate (Packard Instruments B. V., Groningen, The Netherlands). The total amount of added radioligand was measured in Packard 24-well Barex plates (Packard Instruments B. V., Groningen, The Netherlands) in the presence of Microscint™ 20 scintillation fluid (Packard Bioscience, Meriden, Conn., USA).

Buffer

The binding buffer consisted of 20 mM HEPES, 150 mM NaCl, 10 mM MgCl₂, and 1 mM, EDTA, pH 7.4.

Methods

Membrane Preparation

Cells were grown to approximately 90% confluence on 24.5×24.5 mm culture dishes. The medium was aspirated, and after rinsing with ice-cold PBS, the cells were scraped off using 25 ml Tris buffer (50 mM Tris-HCl, 1 mM EDTA, 1 mM EGTA, pH 7.4) and a window scraper. The cells were then broken with a Polytron homogeniser, and remaining particulate matter was removed by low-speed centrifugation, 1000×g for 5 min. Finally, the membranes were collected by high-speed centrifugation (20 000×g), suspended in binding buffer, and frozen in aliquots at −70° C.

Radioligand Binding

Frozen cell membranes were thawed, immediately rehomogenized with a Polytron homogenizer, and coupled to SPA wheat germ agglutinin beads (Amersham Life Sciences, Cardiff, England) for 30 min under continuous shaking of the tubes. After coupling, the beads were centrifuged for 10 minutes at 1000 g, and subsequently suspended in 20 ml of binding buffer per 96-well plate The binding reaction was then initiated by adding radioligand and test compounds to the bead-membrane suspension. Following incubation at room temperature, the assay plates were subjected to scintillation counting.

The original SPA method was followed except for that membranes were prepared from HEK293 cells expressing the human 5-HT₆ receptor instead of from HeLa cells (Dinh D M, Zaworski P G, Gill G S, Schlachter S K, Lawson C F, Smith M W. Validation of human 5-HT₆ receptors expressed in HeLa cell membranes: saturation binding studies, pharmacological profiles of standard CNS agents and SPA development. (The Upjohn Company Technical Report 7295-95-064 1995;27 December). The specific binding of [³H]-LSD was saturable, while the non-specific binding increased linearly with the concentration of added radioligand. [³H]-LSD bound with high affinity to 5-HT₆ receptors. The K_{d} value was estimated to 2.6±0.2 nM based on four separate experiments.

The total binding at 3 nM of [³H]-LSD, the radioligand concentration used in the competition experiments, was typically 6000 dpm, and the specific binding more than 70%. 5-HT caused a concentration dependent inhibition of [³H]-LSD binding with an over all average Ki value of 236 nM when tested against two different membrane preparations. The inter assay variability over three experiments showed a CV of 10% with an average Kᵢ values of 173 nM (SD 30) and a Hill coefficient of 0.94 (SD 0.09). The intra assay variation was 3% (n=4). All unlabelled ligands displaced the specific binding of [³H]-LSD in a concentration-dependent manner, albeit at different potencies. The rank order of affinity for the 5-HT₆ receptor of reference compounds was methiothepin (Ki 2 nM)>mianserin (190 nM) ≈5-HT (236 nM)>methysergide (482 nM)>mesulergine (1970 nM).

Protein Determination

Protein concentrations were determined with BioRad Protein Assay (Bradford M M. A rapid and sensitive method for the quantitation of microgram quantities of protein utilizing the principle of protein-dye binding. Anal. Biochem. 1976;72:248-54). Bovine serum albumin was used as standard.

Scintillation Counting

The radioactivity was determined in a Packard TopCount™ scintillation counter (Packard Instruments, Meriden, Conn., USA) at a counting efficiency of approximately 20%. The counting efficiency was determined in separate sets of experiments.

Saturation Experiments

At least 6 concentrations in duplicates of radioligand (0.1-20 nM of [³H]-LSD) were used in saturation experiments. The specific binding was calculated as the difference between total binding and non-specific binding, which was determined as the binding of radioligand in the presence of 5 μM lisuride. Bₘₐₓ and the dissociation constant, K_{d}, were determined from the non-linear regression analysis using equation 1. Lᵤ is the unbound concentration of radioligand, and is y is the amount bound.

$$y = \frac{{B}_{max} \cdot Lu}{Lu + Kd} \qquad ( equation 1 )$$

Competition Experiments

Total- and non-specific binding of radioligand was defined in eight replicates of each. Samples containing test compound were run in duplicate at 11 concentrations. Incubations were carried out at room temperature for 3 hours. The IC₅₀ value, i.e. the concentration of test compound that inhibited 50% of the specific binding of radioligand, was determined with non linear regression analysis and the Kᵢ value was calculated using equation 2 [Cheng Y. C. Biochem. Pharmacol. 22, 3099-3108, 1973].

$$Ki = \frac{{IC}_{50}}{1 + \frac{L}{{K}_{d}}} \\ L = concentration of radioligand \\ {K}_{d} = Affinity of radioligand \qquad ( equation 2 )$$

(b) 5-HT₆ Intrinsic Activity Assay

Antagonists to the human 5-HT₆ receptor were characterized by measuring inhibition of 5-HT induced increase in cAMP in HEK 293 cells expressing the human 5-HT₆ receptor (see Boess et al. (1997) Neuropharmacology 36: 713-720). Briefly, HEK293/5-HT₆ cells were seeded in polylysine coated 96-well plates at a density of 25,000/well and grown in DMEM (Dulbecco's Modified Eagle Medium) (without phenol-red) containing 5% dialyzed Foetal Bovine Serum for 48 h at 37° C. in a 5% CO₂ incubator. The medium was then aspirated and replaced by 0.1 ml assay medium (Hanks Balance Salt Solution containing 20 mM HEPES, 1.5 mM isobutylmethylxanthine and 1 mg/ml bovine serum albumin). After addition of test substances, 50 μl dissolved in assay medium, the cells were incubated for 10 min at 37° C. in a 5% CO₂ incubator. The medium was again aspirated and the cAMP content was determined using a radioactive cAMP kit (Amersham Pharmacia Biotech, BIOTRAK RPA559). The potency of antagonists was quantified by determining the concentration that caused 50% inhibition of 5-HT (at [5-HT]=8 times EC₅₀) evoked increase in cAMP, using the formula IC_{50,corr}=IC₅₀/(1+[5HT]/EC₅₀).

The compounds in accordance with the invention have a selective affinity to human 5-HT₆ receptors with Kᵢ and IC_{50,corr} values between 0.5 nM and 5 μM or display a % inhibition of [³H]-LSD≧20% at 50 nM and are antagonists, agonists or partial agonists at 5-HT₆. The compounds show good selectivity over human cloned 5-HT₁ₐ, 5-HT_{1b}, 5-HT₂ₐ, 5-HT_{2b}, and 5-HT_{2c} receptors.

TABLE 3
Binding affinity (Ki) at the h 5-HT₆ receptor

| EXAMPLE | Ki (nM) |
|---|---|
| 1 | 140 |
| 3 | 160 |
| 5 | 230 |
| 6 | 356 |
| 7 | 184 |
| 8 | 94 |

(c) In vivo Assay of Reduction of Food Intake

For a review on serotonin and food intake, see Blundell, J. E. and Halford, J. C. G. (1998) Serotonin and Appetite Regulation. Implications for the Pharmacological Treatment of Obesity. CNS Drugs 9:473-495.

Obese (ob/ob) mouse is selected as the primary animal model for screening as this mutant mouse consumes high amounts of food resulting in a high signal to noise ratio. To further substantiate and compare efficacy data, the effect of the compounds on food consumption is also studied in wild type (C57BL/6J) mice. The amount of food consumed during 15 hours of infusion of compounds is recorded.

Male mice (obese C57BL/6JBom-Lep^{ob} and lean wild-type C57BL/6JBom; Bomholtsgaard, Denmark) 8-9 weeks with an average body weight of 50 g (obese) and 25 g (lean) are used in all the studies. The animals are housed singly in cages at 23±1° C., 40-60% humidity and have free access to water and standard laboratory chow. The 12/12-h light/dark cycle is set to lights off at 5 p.m. The animals are conditioned for at least one week before start of study.

The test compounds are dissolved in solvents suitable for each specific compound such as cyclodextrin, cyclodextrin/methane sulphonic acid, polyethylene glycol/methane sulphonic acid, saline. Fresh solutions are made for each study. Doses of 30, 50 and 100 mg kg⁻¹ day⁻¹ are used. The purity of the test compounds is of analytical grade.

The animals are weighed at the start of the study and randomized based on body weight. Alzet osmotic minipumps (Model 2001D; infusion rate 8 μl/h) are used and loaded essentially as recommended by the Alzet technical information manual (Alza Scientific Products, 1997; Theeuwes, F. and Yam, S. I. Ann. Biomed. Eng. 4(4). 343-353, 1976). Continuous subcutaneous infusion with 24 hours duration is used. The minipumps are either filled with different concentrations of test compounds dissolved in vehicle or with only vehicle solution and maintained in vehicle pre-warmed to 37° C. (approx. 1 h). The minipumps are implanted subcutaneously in the neck/back region under short acting anesthesia (metofane/enflurane). This surgical procedure lasts approximately 5 min.

The weight of the food pellets are measured at 5 p.m. and at 8 p.m. for two days before (baseline) and one day after the implantation of the osmotic minipumps. The weigh-in is performed with a computer assisted Mettler Toledo PR 5002 balance. Occasional spillage is corrected for. At the end of the study the animals are killed by neck dislocation and trunk blood sampled for later analysis of plasma drug concentrations.

The plasma sample proteins are precipitated with methanol, centrifuged and the supernatant is transferred to HPLC vials and injected into the liquid chromatography/mass spectrometric system. The mass spectrometer is set for electrospray positive ion mode and Multiple Reaction Monitoring. A linear regression analysis of the standards forced through the origin is used to calculate the concentrations of the unknown samples.

Food consumption for 15 hours is measured for the three consecutive days and the percentage of basal level values is derived for each animal from the day before and after treatment. The values are expressed as mean ±SD and ±SEM from eight animals per dose group. Statistical evaluation is performed by Kruskal-Wallis one-way ANOVA using the percent basal values. If statistical significance is reached at the level of p<0.05, Mann-Whitney U-test for statistical comparison between control and treatment groups is performed.

The compounds according to the invention show an effect (i.e., reduction of food intake) in the range of 5-200 mg/kg/d.

1. A compound of the Formula (I) 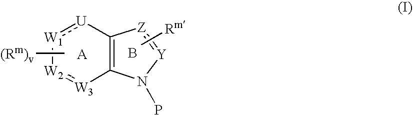
wherein:
v is 1 or 2 and P is selected from a substituent of Formula (II) and Formula (III); 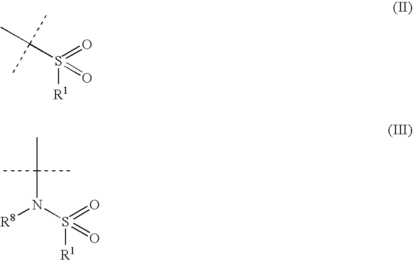
or P may also be selected from H or C₁₋₆-alkyl provided that R^{m} is selected from
—NHSO₂R¹¹, —SO₂NR⁸R¹¹ or —S(O)ₑR¹¹, wherein R¹¹ is selected from aryl and heteroaryl and where e is 0, 1, 2 or 3, v is 1 and R^{m′} is H; [symbol]
represents a single bond or a double bond, with the proviso that both [symbol] represent double bonds or that both [symbol] represent single bonds;
W₁, W₂, W₃, Z and Y are each a carbon atom; or
one of W₁, W₂, W₃, Z and Y is a nitrogen atom, while the remainder being carbon atoms,
provided that both [symbol] in Formula (I) represent single bonds;
U is selected from CHR⁴, CR⁴ and CR⁴R^{4′}, provided that when the dotted line connecting W₁ and U is a double bond, then U is CR⁴; and further provided that when the dotted line connecting W₁ and U is a single bond, then U is selected from CHR⁴ and CR⁴R^{4′};
R¹ is selected from:
(a) C₁₋₆-alkyl,
(b) C₁₋₆-alkoxy-C₁₋₆-alkyl,
(c) C₃₋₆-alkenyl,
(d) hydroxy-C₁₋₆-alkyl,
(e) halo-C₁₋₆-alkyl,
(f) aryl,
(g) arylcarbonylmethyl,
(h) aryl-C₂₋₆-alkenyl,
(i) aryl-C₁₋₆-alkyl,
(j) C₃₋₇-cycloalkyl,
(k) heteroaryl,
(l) 4-piperidinyl,
(m) N-substituted 4-piperidinyl, wherein the substituents are selected from C₁₋₆-alkyl, aryl, heteroaryl, aryl-C₁₋₆-alkyl and heteroaryl-C₁₋₆-alkyl,
(n) heteroaryl-C₁₋₆-alkyl,
wherein any heteroaryl or aryl residue, alone or as part of another group, is optionally substituted, independently, in one or more positions with substituents having the values as defined for R^{m} and R^{m′};
R^{m} and R^{m′} are each independently selected from:
(a) hydrogen,
(b) halogen,
(c) C₁₋₆-alkyl,
(d) hydroxy,
(e) C₁₋₆-alkoxy,
(f) C₂₋₆-alkenyl,
(g) phenyl,
(h) phenoxy,
(i) benzyloxy,
(j) benzoyl,
(k) —OCF₃,
(l) —CN,
(m) hydroxy-C₁₋₆-alkyl,
(n) halo-C₁₋₆-alkyl,
(o) —NR¹⁰R¹⁰,
(p) —NO₂,
(q) —CONR¹⁰R¹⁰,
(r) —NHSO₂R¹¹,
(s) —NR⁸COR¹¹,
(t) —SO₂NR⁸R¹¹,
(u)—C(═O)R¹¹,
(v) C₁₋₆-alkoxycarbonyl,
(w) —S(O)ₑR¹¹, wherein e is 0, 1, 2 or 3,
(x) —SCF₃,
(y) —CHF═CH₂,
(aa) —OCF₂H, or
(ab) ethynyl;
and with the proviso that, R^{m,} is attached to a carbon atom in ring B; and
with the further proviso that when one of W₁, W₂ and W₃ in Formula (I) is a nitrogen atom and both [symbol] represent single bonds the said nitrogen atom is attached to R^{m}, wherein R^{m} is selected from hydrogen or C₁₋₄-alkyl and v is 1; and
with the further proviso that when W₁, W₂ and W₃ in Formula (I) are each a carbon atom and both [symbol] represent single bonds, R^{m} is selected from hydrogen or methyl; and
with the further proviso that when R^{m} or R^{m′}, as substituents on ring A and B in Formula (I), are selected from phenyl, phenoxy, benzyloxy and benzoyl, the phenyl or aryl ring thereof may be optionally substituted by C₁₋₄-alkyl, halogen, C₁₋₄-alkoxy, C₁₋₄-alkylthio, trifluoromethyl, hydroxymethyl or cyano;
wherein R^{m} and R⁴ may be linked to each other to form a fused substituent of Formula (IV) provided that R^{m} is attached to W₁: 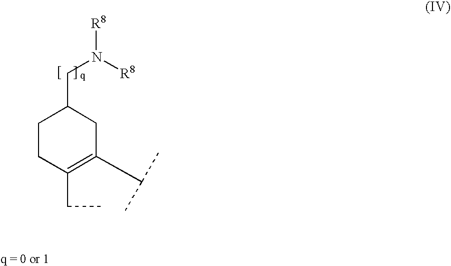
when U is CR⁴ or CHR⁴, R⁴ is a group selected from: 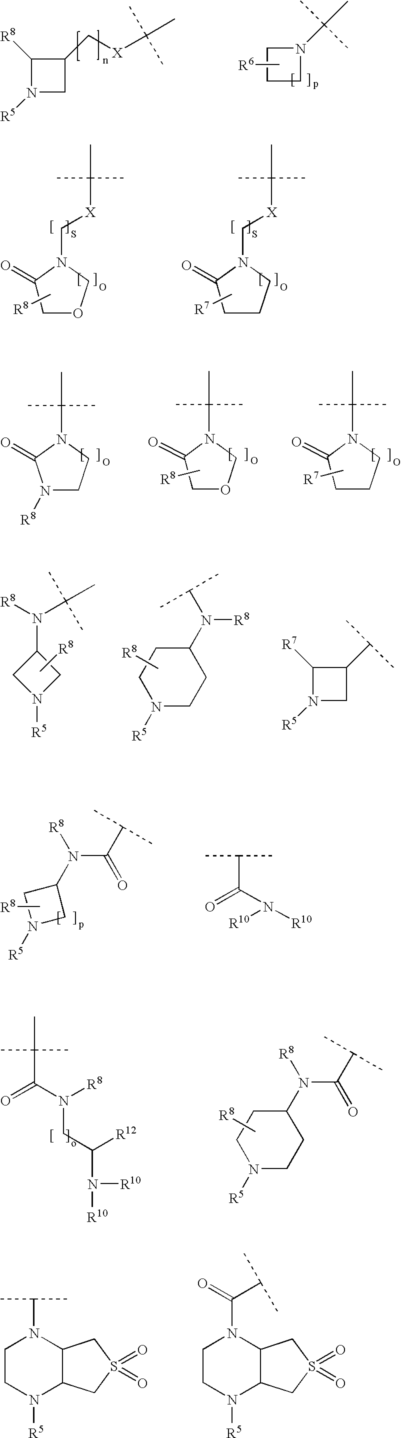 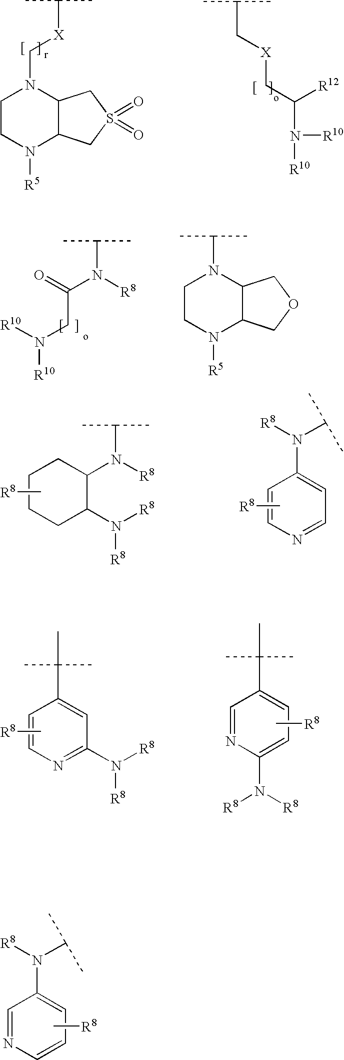
wherein:
n=0, 1, or 2,
o=1 or 2,
p=1, 2, 3, or 4,
r=2 or 3,
s=1, 2 or 3;
when U is CHR⁴, R⁴ is additionally selected from the following groups: 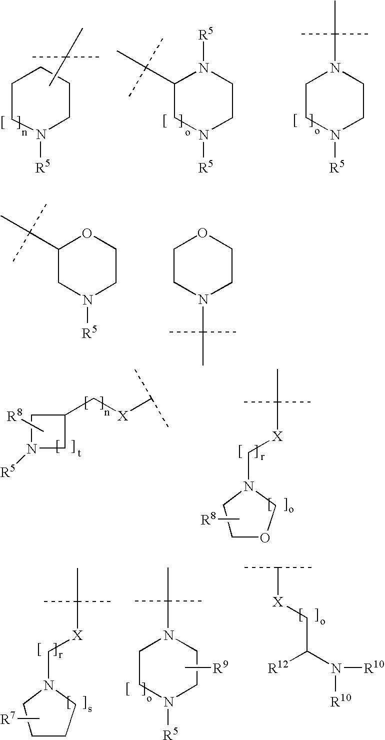
wherein:
n=0,1 or 2,
o=1 or 2,
t=2, 3 or 4,
r=2 or 3,
s=1, 2 or 3;
wherein X is selected from O, NR⁸ and S;
wherein R⁵ is independently a group selected from:
(a) hydrogen,
(b) C₁₋₆-alkyl,
(c) 2-cyanoethyl,
(d) hydroxy-C₂₋₆-alkyl,
(e) C₃₋₆-alkenyl,
(f) C₃₋₆-alkynyl,
(g) C₃₋₇-cycloalkyl,
(h) C₃₋₇-cycloalkyl-C₁₋₄-alkyl,
(i) C₁₋₆-alkoxy-C₂₋₆-alkyl,
(j) aryl-C₁₋₆-alkyl,
(k) heteroaryl-C₁₋₆-alkyl,
(l) 3,3,3-trifluoropropyl,
wherein any aryl and heteroaryl residue may be optionally substituted with C₁₋₄-alkyl, halogen, C₁₋₄-alkoxy, C₁₋₄-alkylthio, trifluoromethyl or cyano;
R⁶ is selected from:
(a) hydrogen,
(b) C₁₋₄-alkyl,
(c) hydroxy-C₁₋₄-alkyl,
(d) C₁₋₄-alkoxy-C₁₋₄-alkyl,
(e) halo-C₁₋₄-alkyl,
(f) —NR⁸R⁸, provided that the said —NR⁸R⁸ group is not attached to a carbon atom adjacent to a ring nitrogen atom,
(g) —CO—NR⁸R⁸;
(h) hydroxy, provided that the said hydroxy group is not attached to a carbon atom adjacent to a ring nitrogen atom;
R⁷ is selected from:
(a) hydrogen,
(b) C₁₋₄-alkyl,
(c) hydroxy-C₁₋₄-alkyl, or
(d) C₁₋₄-alkoxy-C₁₋₄-alkyl,
(e) hydroxy, provided that the said hydroxy group is not attached to a carbon atom adjacent to a heterocyclic ring nitrogen atom and that the said hydroxy group is attached to a heterocyclic ring not substituted with oxo;
R⁸ is each independently selected from:
(a) hydrogen, or
(b) C₁₋₆-alkyl,
R⁹ is selected from:
(a) hydrogen,
(b) C₁₋₄-alkyl, wherein one or two groups may be present at any carbon atom, or when two groups are present at the same carbon atom they may together form a cyclopropane ring,
(c) hydroxy-C₁₋₄-alkyl,
(d) C₁₋₄-alkoxy-C₁₋₄-alkyl,
(e) halo-C₁₋₄-alkyl,
R¹⁰ is each independently selected from:
(a) hydrogen,
(b) C₁₋₆-alkyl,
(c) hydroxy-C₂₋₄-alkyl,
(d) C₃₋₇-cycloalkyl, or
(e) C₁₋₄-alkoxy-C₂₋₄-alkyl,
wherein the two R¹⁰ groups together with the nitrogen to which they are attached form a heterocyclic ring; and when the two R¹⁰ groups form a piperazine ring, the nitrogen of the piperazine ring that allows the substitution may be substituted with a group selected from R⁵;
R¹¹ is selected from:
(a) C₁₋₆-alkyl,
(b) aryl, or
(c) heteroaryl,
wherein aryl and heteroaryl may be optionally substituted with C₁₋₄-alkyl, halogen, C₁₋₄-alkoxy, C₁₋₄-alkylthio, trifluoromethyl, hydroxymethyl or cyano;
R¹² is selected from:
(a) hydrogen, or
(b) methyl;
when U is R⁴R^{4′}, R⁴ and R⁴ are linked to each other to form a heterocyclic ring selected from pyrrolidine or piperidine, wherein the N atom may be substituted by a group selected from R⁵; and pharmaceutically acceptable salts, hydrates, solvates, geometrical isomers, tautomers, optical isomers, and prodrug forms thereof.

2. The compound according to claim 1, wherein
P is selected from 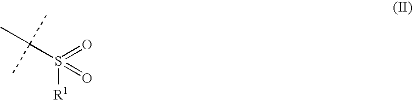
each of W₁, W₂, W₃, Z and Y is a carbon atom provided that both [symbol] in Formula (I) represent double bonds; or
one of W₁, W₂, W₃, Z and Y is a nitrogen atom, while the remainder being carbon atoms, provided that both [symbol] in Formula (I) represent single bonds;
U is selected from CHR⁴, CR⁴ and CR⁴R^{4′}, provided that when the dotted line connecting W₁ and U is a double bond, then U is CR⁴; and further provided that when the dotted line connecting W₁ and U is a single bond, then U is selected from CHR⁴ and CR⁴R^{4′};
R¹ is selected from:
(f) aryl,
(h) aryl-C₂₋₆-alkenyl,
(i) aryl-C₁₋₆-alkyl,
(j) C₃₋₇-cycloalkyl,
(k) heteroaryl,
(n) heteroaryl-C₁₋₆-alkyl,
wherein any heteroaryl or aryl residue, alone or as part of another group, is optionally substituted, independently, in one or more positions with substituents having the values as defined for R^{m} and R^{m′;}
R^{m} and R^{m′} are each independently selected from:
(a) hydrogen,
(b) halogen,
(c) C₁₋₆-alkyl,
(d) hydroxy,
(e) C₁₋₆-alkoxy,
(f) C₂₋₆-alkenyl,
(k) —OCF₃,
(l) —CN,
(m) hydroxy-C₁₋₆-alkyl,
(n) halo-C₁₋₆-alkyl,
(o) —NR¹⁰R¹⁰,
(q) —CONR¹⁰R¹⁰,
(r) —NHSO₂R¹¹,
(s) —NR⁸COR¹¹,
(t) —SO₂NR⁸R¹¹,
(u) —C(═O)R¹¹,
(w) —S(O)ₑR¹¹, wherein e is 0, 1, 2 or 3,
(x)—SCF₃,
(y) —CHF═CH₂,
(aa) —OCF₂H, or
(ab) ethynyl;
and with the proviso that, R^{m,} is attached to a carbon atom in ring B; and
with the further proviso that when one of W₁, W₂ and W₃ in Formula (I) is a nitrogen atom and both [symbol] represent single bonds the said nitrogen atom is attached to R^{m}, wherein R^{m} is selected from hydrogen or C₁₋₄-alkyl and v is 1; and
with the further proviso that when W₁, W₂ and W₃ in Formula (I) are each a carbon atom and both [symbol] represent single bonds, R^{m} is selected from hydrogen or methyl; and
with the further proviso that when R^{m} and R^{m,} are substituents on ring A and B, then R^{m} and R^{m,} are independently selected from: hydrogen, halogen, methyl, methoxy, trifluoromethyl, hydroxymethyl or cyano;
when U is CR⁴ or CHR⁴, R⁴ is a group selected from: 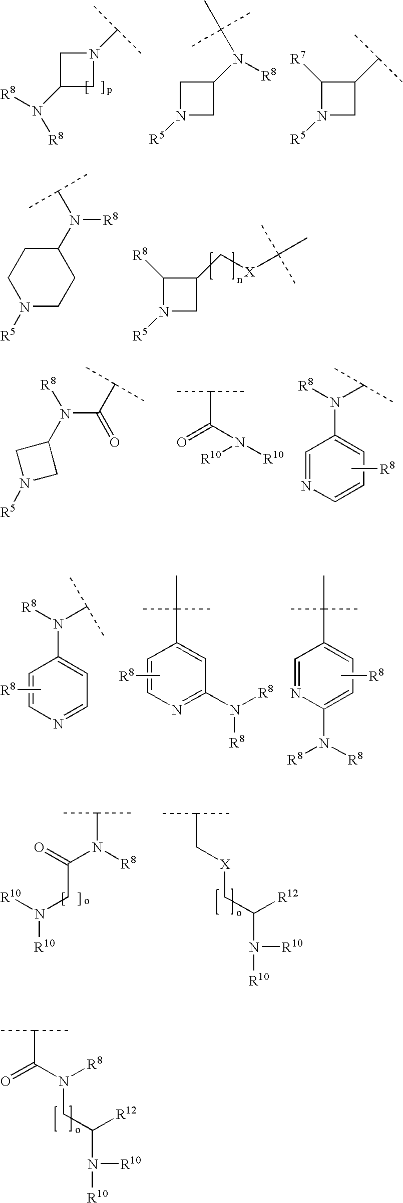
wherein
n=0, 1, or 2,
o=1 or 2,
p=1, 2, 3, or 4,
when U is CHR⁴, R⁴ is additionally selected from the following groups: 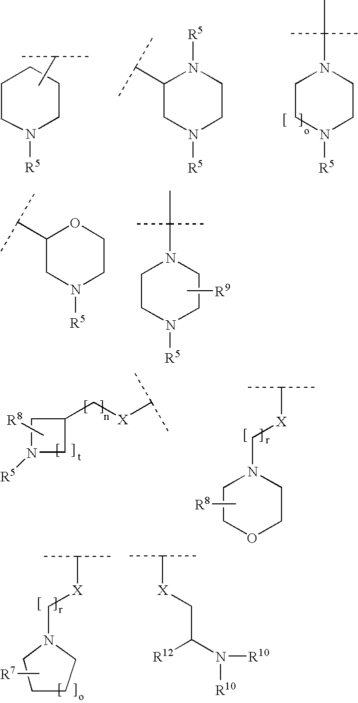
wherein
n=0, 1 or 2,
0=1 or 2,
t=2, 3 or 4,
r=2 or 3,
wherein X is selected from O and NR⁸;
wherein R⁵ is independently a group selected from:
(a) hydrogen,
(b) C₁₋₆-alkyl,
(c) 2-cyanoethyl,
(d) hydroxy-C₂₋₄-alkyl,
(e) C₃₋₆-alkenyl,
(h) C₃₋₇-cycloalkyl-C₁₋₄-alkyl, or
(i) C₁₋₄-alkoxy-C₂₋₄-alkyl,
R⁷ is selected from:
(a) hydrogen,
(b) C₁₋₄-alkyl,
(c) hydroxy-C₁₋₂-alkyl, or
(d) C₁₋₂-alkoxy-C₁₋₂-alkyl;
R⁸ is each independently selected from:
(a) hydrogen, or
(b) C₁₋₆-alkyl,
R⁹ is selected from:
(a) hydrogen,
(b) C₁₋₄-alkyl, wherein one or two groups may be present at any carbon atom, or when two groups are present at the same carbon atom they may together form a cyclopropane ring,
(c) hydroxy-C₁₋₂-alkyl,
(d) C₁₋₂-alkoxy-C₁₋₂-alkyl,
(e) halo-C₁₋₂-alkyl,
R¹⁰ is each independently selected from:
(a) hydrogen,
(b) C₁₋₄-alkyl,
(c) hydroxy-C₂₋₄-alkyl
wherein the two R¹⁰ groups together with the nitrogen to which they are attached form a heterocyclic ring; and when the two R¹⁰ groups form a piperazine ring, the nitrogen of the piperazine ring that allows the substitution may be substituted with a group selected from R⁵;
R¹¹ is selected from:
(a) C₁₋₄-alkyl
R¹² is selected from:
(a) hydrogen, or
(b) methyl;
when U is R⁴R^{4′}, R⁴ and R^{4′} are linked to each other to form a heterocyclic ring selected from pyrrolidine or piperidine, wherein the N atom may be substituted by a group R⁵ selected from:
(a) hydrogen,
(b) C₁₋₄-alkyl,
(d) hydroxy-C₂₋₄-alkyl,
(i) C₁₋₄-alkoxy-C₂₋₄-alkyl,
(k) 2-cyanoethyl.

3. The compound according to claim 1 or 2, wherein
Ar is selected from phenyl, naphthyl, and thienyl, which group Ar is optionally substituted by halogen, methyl, methoxy.

4. The compound according to claim 1 which is selected from 4′-Methyl-1′-(2-naphthylsulphonyl)-1′,4′,5′,6′-tetrahydrospiro{piperidine-2,7′-pyrrolo[3,2-b]pyridine} hydrochloride,
4′-Methyl-1′-(4-bromophenylsulphonyl)-1′,4′,5′,6′-tetrahydrospiro {piperidine-2,7′-pyrrolo[3,2-b]pyridine} hydrochloride,
4′-Methyl-1′-(5-bromo-2-thienylsulphonyl)-1′,4′,5′,6′-tetrahydrospiro {piperidine-2,7′-pyrrolo[3,2-b]pyridine} hydrochloride,
4′-Methyl-1′-(2-thienylsulphonyl)-1′,4′,5′,6′-tetrahydrospiro{piperidine-2,7′-pyrrolo[3,2-b]pyridine} hydrochloride
N-(1-Benzenesulfonyl-1H-indol-4-yl)-2-(2-hydroxy-ethylamino)-acetamide, and
1-Benzenesulfonyl-1H-indol-4-yl)-pyridin-4-yl-amine,
N-(4-piperazin-1-yl-1H-indol-1-yl)benzenesulfonamide hydrochloride, and
3-(Phenylsulfonyl)-6,7,8,9-tetrahydro-3H-benzo[e]indol-8-amine trifluoroacetate.

5. A process for the preparation of a compound according to claim 1, comprising the following steps:
(a) reaction of 2-(2-ethylamino)pyrrole and 1-methylpiperazine-4-one to give 4′-methyl-1′,4′,5′,6′-tetrahydrospiro{piperidine-2,7′-pyrrolo[3,2-b]pyridine}; and
(b) reaction of the product from step a) with an arylsulphonyl chloride in the presence of a base.

6. A process for the preparation of a compound according to claim 1, comprising the following steps:
(c) reaction of 1-benzensulfonyl-1H-indol-4-ylamine and bromoacetyl bromide and further reaction with ethanolamine.

7. A process for the preparation of a compound according to claim 1, comprising the following steps:
(d) reductive amination of 3-(toluene-4-sulfonyl)-6,9-dihydro-3H, 7H-benzo[e]indol-8-one in the presence of sodium cyanoborohydride and ammonium acetate.

8. (canceled)

9. (canceled)

10. (canceled)

11. A pharmaceutical formulation comprising a compound according to claim 1 or claim 2, in combination with a pharmaceutically acceptable diluent or carrier.

12. (canceled)

13. (canceled)

14. A method for the prophylaxis or treatment of a 5-HT₆ receptor-related disorder, to achieve reduction of body weight and of body weight gain, which comprises administering to a subject in need of such treatment an effective amount of a compound according to claim 1 or claim 2.

15. The method according to claim 14, wherein the disorder is selected from obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder, withdrawal from drug abuse, neurodegenerative diseases characterized by impaired neuronal growth, and pain.

16. A method for modulating 5-HT₆ receptor activity, which comprises administering to a subject in need of such treatment an effective amount of a compound according to claim 1 or claim 2.

17. (canceled)

18. (canceled)

19. A cosmetic composition comprising a compound according to claim 1 or claim 2 as active ingredient, in combination with a cosmetically acceptable diluent or carrier.

20. (canceled)

21. A method for the treatment or prevention of a 5-HT₆ receptor-related disorder, the method comprising administering a composition comprising the compound of claim 1.

22. The method of claim 21 wherein the disorder is selected from the group consisting of: obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder, withdrawal from drug abuse, neurodegenerative diseases characterized by impaired neuronal growth, and pain.

23. A method for the treatment or prevention of a 5-HT₆ receptor-related disorder, the method comprising administering the pharmaceutical composition of claim 11.

24. The method of claim 23 wherein the disorder is selected from the group consisting of: obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder, withdrawal from drug abuse, neurodegenerative diseases characterized by impaired neuronal growth, and pain
